(19) United States
(12) Patent Application Publication
(10) Pub. No.: US 2026/0284333 A1
(43) Pub. Date: Sep. 24, 2026

(54) NEBULIZER AND CONTAINER FOR A NEBULIZER

(71) Applicant: NEUTEC INHALER ILAC SANAYI VE TICARET ANONIM SIRKETI, Istanbul (TR)

(72) Inventor: Mahmut BILGIC, Istanbul (TR)

(21) Appl. No.: 19/471,634

(22) PCT Filed: Apr. 6, 2023
(86) PCT No.: PCT/IB2023/053508

§ 371 Date: Oct. 1, 2025

(51) Int. Cl. A61M 15/00; A61M 11/00

(52) U.S. Cl. CPC A61M 15/0073; A61M 11/006; A61M 15/0026; A61M 2202/0007; A61M 2202/04; A61M 2205/12

(57) ABSTRACT

The invention relates firstly to a nebulizer (1) for a fluid (3) located in a container (2), having a housing (4), the housing (4) having a mouthpiece (7) and a receiving space (112) for the container (2), which container (2) can be inserted replaceably into the housing (4), wherein further for the preparation of an atomizing process a lower housing part (6) is rotatable relative to an upper housing part (5) having the mouthpiece (7), a counter (16 ) is provided in the lower housing part (6), for counting atomizing operations carried out with an inserted container (2), and the rotation of the housing parts (5, 6) relative to one another can be used to count the atomizing operations, the counter (16) having a sleeve part (51) which is provided on an outer surface (52) with characters (53) representing atomizing operations counted and with a toothing (57) for toothing engagement, a transmission part (43) further being provided which engages in this toothing (57). In order to further improve the design of a nebulizer of the type in question, it is proposed that the toothing engagement is provided in the transverse direction to a sleeve axis (c) and that the transmission part (43) is arranged and formed for direct cooperation with both the upper housing part (5) and the lower housing part (6) as well as with the sleeve part (51).

### FIELD OF TECHNOLOGY

The invention relates to a nebulizer for a fluid located in a container, having a housing, the housing having a mouthpiece and a receiving space for the container, which can be inserted replaceably into the housing, it further being possible for a lower housing part to be rotated relative to an upper housing part having the mouthpiece in preparation for an atomization process, a counter being furthermore provided in the lower housing part, for counting atomizing operations carried out with an inserted container, and the rotation of the housing parts relative to one another can be used for counting the atomizing operations, wherein the counter comprises a sleeve part which is provided on an outer surface with characters representing the counts and with a toothing for toothing engagement, wherein furthermore a transmission part is provided which engages in this toothing.

The invention further relates to a nebulizer for a fluid located in a container, comprising a housing, the housing having a mouthpiece and a receiving space for the container, which is exchangeably insertable into the housing, wherein furthermore for the preparation of an atomizing process a lower housing part is rotatable relative to an upper housing part having the mouthpiece, wherein furthermore a counter is provided for counting atomizing processes carried out with a container, wherein furthermore the counter has a sleeve part which is insertable into the housing, for preparing an atomizing operation, a lower housing part being rotatable relative to an upper housing part having the mouthpiece, a counter being further provided for counting atomizing operations performed with an inserted container, the counter further comprising a sleeve part provided on an outer surface with characters representing the counts.

In addition, the invention relates to a nebulizer for a fluid located in a container, with a housing, the housing having a mouthpiece, and a receiving space for the container which can be inserted replaceably into the housing, wherein a removed container in the housing can no longer be placed in a position of use.

Also, the invention relates to a container for a nebulizer.

### PRIOR ART

Nebulizers as well as containers for a nebulizer are known, for example, from WO 1997/012687 A1 (U.S. Pat. No. 5,964,416 A) or also from WO 1991/014468 A1 (U.S. Pat. No. 5,497,949 A), WO 2007/022898 A2 (U.S. Pat. No. 7,823,584 B2) and further, for example, from WO 2017/080895 A1 (US 2017/0128681 A1 ). Furthermore, a metering device with counter has become known, for example, from WO 1997/024586 A1 (U.S. Pat. No. 6,149,054 A) or also from WO 2007/022898A2 (U.S. Pat. No. 7,823,584 B2), and furthermore, for example, also from WO 2015/169428A2 (U.S. Pat. No. 10,099,022 B2).

The container thereby forms, whereby this description also concerns possible parts and modes of operation of the nebulizer according to the invention, a reservoir for an atomizing fluid, whereby such a container is replaceably insertable into the receiving space of the nebulizer. In this regard, the container may have a rigid outer shell, for example made of a metal material, such as further for example aluminum, and an inner bag in which the fluid is stored. Preferably, the fluid is stored in the container without pressure, i.e. without propellant gas or the like.

Specifically, as a result of the rotational displacement, the container is lowered within the housing, along with a hollow piston located therein. During the lowering, a spring acting on the container is tensioned. At the same time, fluid flows into a pressure chamber located outside the container and associated with one end of the hollow body. This can be achieved by a negative pressure caused in the pressure chamber during this process. By means of a release actuation, the container together with the hollow piston located therein can be released for an upward pressure movement. Here, the fluid in the pressure chamber is pressurized and then ejected as a spray mist. For this purpose, the hollow piston can preferably be provided with a check valve at its end associated with the pressure chamber so that it can act as a piston.

Thus, through the preferred rotational displacement of the upper and lower housing parts relative to each other, a predetermined amount of fluid (dosing amount) is brought into a chamber, from which the divided amount of fluid is expelled voluntarily by user intervention and atomized into an aerosol for inhalation by the user. The expulsion of the fluid dose takes place according to a pressurization, which pressure is built up in the course of the relative rotation of the upper and the lower housing part to each other due to tension of a spring. Thereby a pressure of for example about 5 to 200 MPa, further for example 10 to 100 MPa can act on the fluid to be expelled, whereby with each stroke a fluid delivery of about 10 to 50 μl, further for example about 10 to 20 μl, so further for example about 15 μl can take place. When atomized into an aerosol, droplets with an aerodynamic diameter of, for example, up to 20 μm, preferably between about 3 and 10 μm, can be created.

Furthermore, it is known to provide a counter which registers and dis-plays the atomization operations performed. In the case of a nebulizer such as is known from one or more of the aforementioned publications, the counting is carried out essentially at a moment when the fluid dose atomization takes place as a result of pressurization or in the course of twisting of the housing parts relative to one another.

### SUMMARY OF THE INVENTION

In view of the aforementioned prior art, the invention presents the task of further improving a nebulizer of the type in question as well as a container for a nebulizer.

According to a first idea of the invention, a possible solution of the task is given in a nebulizer, in which it is intended that the toothing engagement is given in transverse direction to a sleeve axis and that the transmission part is arranged and formed for direct cooperation with the upper housing part as well as with the lower housing part and also with the sleeve part.

As a result of the proposed design and arrangement of the transmission part, there is an overall advantageous and favorable design of the nebulizer, in particular with regard to the counting mechanism accommodated in the nebulizer. By means of such a transmission part, a reduction in the number of components can be achieved, in particular with regard to the assembly for detecting and counting the atomization processes. Preferably, no further transmitting (intermediate) part is provided between the housing parts, which can be rotated relative to one another, and the transmission part, or between the sleeve part and the transmission part. Accordingly, there is a direct, immediate interaction in these transmission areas.

For example, the transmission part may be rotatably mounted on one of the housing parts in such a way that the other housing part can act on the transmission part during the relative rotation necessary to prepare an atomization process, for example by dragging it along, in such a way that a rotation of the transmission part about a predetermined circumferential angle results for counting (or partial counting).

The further alignment of the toothing engagement between the transmission part and the sleeve part in a direction transverse to the sleeve axis preferably further results in an alignment of a geometric axis of the transmission part in the same alignment as the sleeve part axis. This makes it possible to achieve a compact design of the nebulizer as a whole.

Each fluid ejection process or inhalation process is necessarily preceded by a relative rotation of the housing parts with respect to one another in order to convey a partial fluid quantity into an ejection chamber or into the said pressure chamber, and in order to pressurize and eject this partial fluid quantity via the spring tensioned in the course of the rotation. In an advantageous manner, a count is already carried out in the course of preparation for a fluid ejection process, whereby the relative rotation of the upper and lower housing parts with respect to one another is registered and adopted as a count value.

Furthermore, the corresponding count, i.e. the corresponding action on the counter, can take place immediately at the beginning of the relative rotation, alternatively at the end of the preferably stop-limited relative rotation. In this respect, counting is also possible when an intermediate position is reached between the beginning and the end of the relative rotation of the housing parts with respect to each other.

The counting, or the direct action on the sleeve part via the transmission part, corresponding to a change in the display of the counted atomization processes, can take place at each preferred relative rotation of the housing parts to each other by, for example, 180 degrees, but alternatively also, for example, only at every second atomization process, so further, for example, at each relative rotation of the housing parts to each other by a total of 360 degrees. In addition, according to a further exemplary embodiment, a count or a change in the display of the counted atomization processes can take place, for example, only at every fourth atomization process, so further, for example, after every relative rotation of the housing parts to each other by a total of 720 degrees.

Below, the upper housing part is often also described as the fixed housing part and the lower housing part as the housing part rotatable relative to this fixed upper housing part.

According to a preferred embodiment, the counter can be connected to the lower housing part and thus preferably to the rotatable housing part. Further preferably, the counter is held in the lower housing part so that it cannot be removed by the user or cannot be removed non-destructively.

The counter can have a sleeve part which is provided on an outer surface with characters representing the counts. In particular, as is also preferred, the currently valid character may be readable from the outside through a viewing window. Further preferably, in this respect a character area in front of and/or behind the currently valid character can be readable in the viewing window.

With regard to the characters arranged on the sleeve part, these may be numbers and/or symbols, whereby symbols, for example in the form of dashes, may be provided in an area between two numbers following one another in the circumferential direction. Furthermore, the characters can indicate, starting at zero and in ascending order, the number of atomization processes already carried out or alternatively, as is also preferred, the current number of atomization processes that can still be carried out with this container, descending from an initially displayed maximum number of possible atomization processes.

To change the count value to be displayed, the sleeve part can be rotated in the lower housing part and relative to it about a sleeve part axis or housing axis. In the axial direction, however, the sleeve part is trapped in the housing part.

Further, the sleeve member may have serrations directed transversely to the sleeve member axis, for example, as is also preferred, serrations directed substantially in the radial direction. This may, as is also preferred, be a uniform toothing viewed in the circumferential direction, which serves to drive the rotational displacement of the sleeve part in dependence on the performance of an atomization process, or, as is preferred, in dependence on a preparation for a subsequent atomization process. The housing upper part, which is fixed relative to the housing lower part about the longitudinal axis of the housing, acts indirectly via the transmission part on the toothing of the sleeve part for the stepwise rotational displacement of the latter.

Furthermore, the toothing of the sleeve part may be a toothing pointing radially inwards from a substantially circumferential sleeve part wall.

The aforementioned sleeve axis is preferably the axis about which the sleeve part can rotate, wherein the sleeve axis is further preferably aligned with the longitudinal housing axis and further preferably with a rotational axis of the rotatable housing part. In a further embodiment, the aforementioned longitudinal axis of the housing can simultaneously form the sleeve axis and the axis of rotation of the rotatable housing part.

In addition, according to a further idea of the invention, a further solution to the problem can be provided in a nebulizer having a counter comprising a sleeve part in that the sleeve part cooperates with a locking projection which is guided in the upper housing part so as to be fixed in the direction of rotation and which, in a selected position of the sleeve part, moves into a locking opening of the sleeve part.

Preferably, the sleeve part is further rotated in the circumferential direction of the sleeve part during each active count, which count may occur during each individual atomization process, as well as possibly only during, for example, every second or fourth atomization process. This rotation may be prevented due to a locking engagement of the locking projection in the locking opening of the sleeve part. The sleeve part, and thus preferably the entire counter, can thereby be blocked, and further preferably a blocking of the relative rotational displacement of the housing parts with respect to each other can also be achieved by this blocking. Accordingly, in such a blockade position of the sleeve part, the nebulizer can (initially) no longer be used for ejecting a fluid dose. The nebulizer is blocked with regard to its functionality.

The arrangement and preferably linear guidance along the housing axis of the locking projection in the upper, fixed housing part and the formation of the locking opening in the lower, rotatable housing part in the blocking position result in an almost direct (via the sleeve part) rotational lock between the housing parts. This lock can, as is also preferred, be selected in such a way that immediately after the locking projection has moved into the locking opening, no further relative rotational displacement of the housing parts with respect to one another is permitted.

The selected position in which the locking projection interacts with the locking aperture can, as is also preferred, be the counter position representing an—at least approximately—emptied container, so further for example the zero position of the counter.

The blockage provided, preferably in the zero position of the counter, and the blockage of the relative rotation of the housing parts with respect to one another, which preferably goes hand in hand with this, prevent the nebulizer from being used when the container is empty.

The blockage can preferably be removed on its own, by replacing the—at least approximately—empty container with a new, filled container, while the counter remains in the housing.

In a nebulizer of the type in question, in which a removed container cannot be (re)placed in a position of use in the housing, a further possible solution to the problem can be provided by the container being provided with a fixedly connected extension arm and by the extension arm, in the course of insertion of the container into the housing, cooperating with a projection fixed to the housing to move an insertion locking part into an activated position.

With regard to a container for a nebulizer, according to a further idea of the invention, the task can be solved in that the container is provided with a fixedly connected cantilever and that the cantilever comprises an insertion locking part.

The insertion locking part, which is moved into an activated position, advantageously prevents re-insertion and reaching of the position of use in the housing of a container which has already been used, correspondingly already inserted in a nebulizer. For this purpose, the extension provided on the container preferably already cooperates with the projection fixed on the housing side in the course of the first insertion of the container—i.e. the insertion of the container before a first use of the same. There is thereby preferably a non-bypassable positive control of the insertion locking part into the activated position. The activated lock is preferably part of the replaceable container or of the boom arranged on this container and thus preferably also replaceable with it. The boom is further preferably connected to the container in such a way that a user cannot separate the boom from the container without destroying it.

A further preferred arrangement of the insertion locking part is one that is protected against manipulation. In particular, the activated position is preferably not removable by the user.

All the features described with regard to the insertion locking part can be realized individually or in combination, insofar as they are not described as individuals, in the insertion locking part given with regard to the container on the fixedly connected cantilever.

According to a preferred embodiment, the extension arm may be a separate plastic part, but connected to the container and preferably not separable from the container by the user in a non-destructive manner.

In an advantageous manner, as is preferred, only one (number word) insertion locking part that can be activated or activated or, in particular, only one (number word) section of the insertion locking part that can be brought into the activated position and can cooperate with the housing in a locking manner can be provided.

The features of the independent claims described above are each essential in themselves as well as in any combination with each other, whereby further features of an independent claim can be combined with the features of a further independent claim or with features of several independent claims, further also with only individual features of one or more of the further independent claims.

Further features of the invention are explained below, also in the figure description, often in their preferred assignment to the subject-matter of claim 1 and/or of the further independent claim(s) or to features of further claims. However, they may also be significant in an assignment to only individual features of claim 1 and/or of the further independent claim(s) or of the respective further claim, or independently in each case.

In one possible embodiment, the transmission part may be formed as a transmission shaft. The transmission shaft is rotatable about a geometric axis of rotation directed in the longitudinal direction of the shaft, for preferred cooperation with the splines of the sleeve part.

The geometric axis of rotation of the transmission shaft can, as is also preferred, be aligned with an axis of rotation of the sleeve part, and can also be aligned with the axis of rotation about which the housing parts of the nebulizer housing can be rotated relative to one another.

The transmission shaft may have regions spaced from each other in two directions of its axis of rotation, forming first and second gear tooth formations. Each gear tooth formation may be provided by a tooth-like formation alone, but alternatively may be provided by arranging a plurality of tooth-like formations, and further optionally by a gear-like formation.

The first tooth formation can be designed to cooperate with the upper and lower housing part and the second tooth formation can be designed to cooperate with the sleeve part. With regard to the first toothed formation, as is also preferred, there can be direct cooperation with the upper and at the same time with the lower housing part, and there can also be direct cooperation with the sleeve part, in particular with the toothing of the sleeve part, with regard to the second toothed formation. The interaction with the upper and/or lower housing part can also be indirect, for example via a housing inner part connected to the respective housing part in a rotationally fixed manner.

It can also be further provided that the lower housing part has a circumferential first support flange for the locking projection, the support flange leaving a passage opening for the locking projection in a selected circumferential region. If necessary, the locking projection can be supported on the supporting flange over at least a partial circumferential region in the course of the relative rotation of the housing parts with respect to one another. Only in a selected circumferential orientation of the sleeve part having the locking opening relative to the passage opening of the support flange is passage of the locking projection for locking the housing parts against one another possible. Preferably, this is the circumferential position in which the locking opening of the sleeve part and the passage opening of the support flange overlap with respect to the direction of travel of the locking projection.

In addition, the sleeve part can have a second support flange which has the locking opening for the locking projection in a selected circumferential region. The second support flange, like the first support flange of the lower housing part described above, serves to support the locking projection in the course of a usual relative rotation of the housing parts with respect to each other, in which the emptying position of the container to be locked by the locking projection is not reached.

According to a further development, the insertion locking part, which can be displaced into an activated position in the course of insertion of the container into the housing, can move in a transverse direction to a longitudinal axis of the nebulizer in the course of such insertion. This movement may be achieved as a result of a corresponding displacement due to a collision with the projection fixed to the housing. Due to the movement transversely to the longitudinal direction of the nebulizer, the insertion locking part, in particular a section of the insertion locking part, is brought into an activated position, which preferably does not allow a renewed insertion of such a container—after a previously performed removal from the housing.

At least part of the insertion locking part can be displaced from the activated position towards a temporary non-activated position against spring force. The spring force can result from a separately provided spring, for example a metal spring, further for example in the form of a leaf spring, leg spring or cylinder compression spring. However, the spring force can also be achieved by a corresponding design of the insertion-locking part itself or by a region of the cantilever that interacts with the insertion-locking part.

This spring-loaded displacement of the insertion-locking part can be used, for example, to achieve non-destructive removal of the container with its extension arm from the housing, wherein the insertion-locking part or a section of the insertion-locking part, in the course of the displacement of the container or of the extension arm during removal from the housing, is initially temporarily displaced back into a non-activated position against spring force as a result of corresponding loading via a housing section and, after removal of this loading via the housing section, moves back into the activated position again as a result of the spring force. Accordingly, an overflowable active position of the insertion locking part can be given in the state of the container inserted into the housing.

In a further preferred embodiment, the insertion locking part is blocked beyond the activated position. Accordingly, no movement of the insertion-locking part or of a section of the insertion-locking part out of the activated position can be achieved in a direction opposite to the possible sprung deflection direction of the insertion-locking part or of a section of the insertion-locking part.

In one possible embodiment, the spring force can be achieved by designing the insertion locking part or a subsection of the insertion locking part as a spring arm. Thus, the insertion locking part can further be manufactured as a whole, for example as a plastic injection-molded part, wherein a subsection, in particular a subsection carrying the latch section that can be displaced into the activated position, is formed as a spring arm by corresponding design.

The spring force can also be formed by a spring arm that is formed separately from the insertion locking part and can be latched to the insertion locking part.

Such a spring arm preferably acts on the insertion locking part only after displacement of the insertion locking part into the activated position, preferably as a result of latching of the insertion locking part with the spring arm in the activated position. Once the latching position between the insertion locking part and the spring arm has been assumed, it is preferably not at the will of the user, and furthermore in particular not toolless and non-destructively removable. Rather, the latching position is permanently adopted after the insertion latching part has been moved into the activated position in the course of inserting the container into the housing.

The spring arm provided separately from the insertion locking part can be formed on the extension arm according to a possible embodiment. On this extension arm, the insertion locking part is further preferably movably held, so that it is further in particular pivotably movable about a geometric axis of rotation, which is preferably transverse to an insertion movement of the container with the extension arm into the housing, further preferably transverse to a housing longitudinal axis.

The locking projection, which is fixed to the direction of rotation and which moves into the locking opening of the sleeve part in a selected position of the sleeve part, serves to make it clear to the user that the container is to be changed. The locking projection may be displaceable to a release position in the course of removing the container from the housing, so as to make a new container usable. This displacement can take place indirectly, for example via a lever or the like provided in the housing, or alternatively directly through the container or a section thereof. Thus, when the container is removed, the counter can be put back into a position from which, for example, atomization processes can be counted again after insertion of a new container. Further preferably, the locking projection is held in a position releasing the locking opening of the sleeve part until the locking position is reached again, for example until a position representing an at least approximately empty container is reached again.

Preferably, such a displacement of the locking projection into the release position re-enables the function of the nebulizer as a whole.

Also, in particular in connection with the rotational release of the sleeve part, but preferably also independently thereof, the sleeve part can be moved into a count start position in the course of a removal of the container from the housing. The counting start position can be a zero position in an ascending counting mode to indicate the atomization processes already carried out, and in a descending counting mode to indicate the atomization processes that can still be carried out, for example a maximum possible number of atomization processes in relation to the container used. Depending on the container size and/or depending on the discharge quantity per fluid dose, the initial counting position can represent, for example, 200, 150, 120, 100, 80, 60 or also 30 or also another maximum possible, preferably integer, number of atomization processes. The sleeve part is reset to the initial counting position by rotating the sleeve part about its axis, further preferably in a direction of rotation which is also assumed during a count.

According to a preferred embodiment, the displacement of the locking projection into the release position and the displacement of the sleeve part into the count start position can be carried out in combination. Thus, the release of the sleeve part according to the backward displacement of the locking projection and the displacement of the sleeve part into the initial counting position can take place simultaneously or alternatively one after the other, but preferably combined in such a way that both measures acting on the sleeve part take place in the course of preferably only one intervention by the user in the same direction. This results in a convenient release and reset of the counter remaining in the housing during container removal.

Thus, further, the container may be provided with the fixedly connected cantilever. This extension arm can be designed to act on the sleeve part for rotation into the counting starting position and/or for displacement of the locking projection into the release position in the course of removal of the container from the housing. The extension arm is preferably rigidly connected to the container. Via this extension arm, if necessary, a partial section thereof can act on the sleeve part, in particular on a section of the circumferential wall for rotation of the sleeve part into the counting starting position. Another section of the cantilever, but possibly the same section, can act, for example, via a housing-side lever to displace the locking projection to the release position. Alternatively, the cantilever can also act directly or indirectly via the sleeve-side support flange on the locking projection to displace the latter to the position releasing the sleeve part. In this way, the locking projection can also be forced out of the locking opening on the sleeve part in the course of the rotational displacement of the sleeve part into the counting starting position.

In a further embodiment, the insertion locking part can be pretensioned into a locking position when the container is inserted. The pretension can be applied by a separately provided spring element. Alternatively, the insertion locking part is designed in such a way that the required pretensioning force results from the insertion locking part itself. For example, the insertion locking part can be a plastic injection-molded part with a section forming a spring arm which carries the locking section, which can be brought into a pretensioned position by the projection fixed to the housing at least in the course of displacement of the container during insertion into the housing and/or removal from the housing. Without being acted upon by the projection, the locking section can be in a spring-released locking position.

In the housing, in particular in the lower fixed housing part, a guide receptacle can be formed for the extension arm, via which guide receptacle the container with the extension arm can be inserted into the housing or guided out of the housing, preferably linearly along the longitudinal axis of the housing. This can further result in an unambiguous assignment orientation of the container relative to the housing in an advantageous manner for the user. An obstacle projection fixed to the housing can be provided in the guide receptacle, which must be overcome in particular when the container is inserted into the housing.

The obstacle projection provided is intended to ensure that only a container that has not yet been inserted into a nebulizer housing can be moved to the proper position of use within the housing. A container that has already been used should no longer be able to be moved into a proper position in the housing that enables an atomization process, regardless of the filling level of the container. This is achieved by the housing-fixed obstruction projection, which cooperates with the insertion locking part when an already used container is inserted, to prevent the container from being moved to the position of use. In this position, an activated insertion locking part preferably engages in a travel path of the cantilever, in which travel path the housing-side obstruction projection basically dips. The container can only be properly inserted into the housing when the insertion locking part is not moved into the activated position.

The insertion locking part preferably enters the activated position as soon as the insertion of an unused container into the housing is completed, as a result of a collision with the projection fixed to the housing. When a container is removed, the insertion-locking part can preferably pass over the housing-fixed obstruction projection with resilient deflection of the insertion-locking part. For this purpose, the insertion locking part can have a deflection slope. By such a design, it can be advantageously achieved in particular that the insertion-locking part does not have any tension in a springable section for the duration of the use of the container. In the opposite direction of displacement, however, when an attempt is made to insert this used container into the housing, such overrunning of the obstruction projection is prevented by design measures that, for example, no deflection can occur as during removal.

The proper use position of the container is preferably the position in which the container is gripped by a clamping fixture on the housing side and a suction tube of the nebulizer is immersed in the container.

With regard to supplementary or alternative features in connection with a container for a nebulizer, the same applies to their claim-related assignment as stated above with regard to the features relating to the nebulizer.

### BRIEF DESCRIPTION OF THE DRAWINGS

The invention is explained below with reference to the enclosed drawing, which, however, represents only examples of embodiments. A part which is explained only with reference to one of the embodiments and is not replaced by another part in a further embodiment due to the special feature highlighted there is thus also described for this further embodiment as a part which is in any case possible. The drawing shows:

FIG. 1 the nebulizer in perspective view;

FIG. 2 another perspective view of the nebulizer after opening a cover cap that exposes a mouthpiece;

FIG. 3 the nebulizer in a perspective exploded view;

FIG. 4 a perspective exploded view of a substantially lower housing area of the nebulizer;

FIG. 5 a perspective exploded view of a substantially upper housing area of the nebulizer;

FIG. 6 an exploded perspective view of a container for insertion into the nebulizer and of a counter that can be assigned to a lower housing inner part;

FIG. 7 the container with an associated container connection part in perspective view;

FIG. 8 an exploded perspective view of the connection area of the container and container connection part;

FIG. 9 the connection area of the container connection part with an associated insertion locking part in perspective view, relating to a first embodiment of the container connection part;

FIG. 10 an another perspective view of the connection area of the container connection part according to FIG. 9;

FIG. 11 an opened insertion area of the nebulizer for inserting a container with a container connection part;

FIG. 12 in a perspective view of a closure cap of the nebulizer;

FIG. 13 the section according to line XIII-XIII in FIG. 1, an intermediate position in the course of inserting a container into the housing;

FIG. 14 the section according to FIG. 13, but concerning the insertion position of the container, before a final arrangement of the closure cap;

FIG. 15 a sectional view according to FIG. 13, concerning the basic position of the nebulizer;

FIG. 16 an another sectional view according to FIG. 13, concerning the preparation position of the nebulizer;

FIG. 17 the Section XVII-XVII in FIG. 16;

FIG. 18 an enlarged view of area XVIII in FIG. 17, relating to an intermediate position with the sectional plane offset;

FIG. 19 the section according to line XIX-XIX in FIG. 18;

FIG. 20 is a subsequent illustration to FIG. 16, concerning the atomization position;

FIG. 21 an exploded perspective view of a release ring with release button and associated intake manifold part;

FIG. 22 an additional explosion perspective view of the components according to FIG. 21;

FIG. 23 an upper housing interior in perspective view;

FIG. 24 the inner part of the housing in a sectional view along sectional plane XXIV in FIG. 23;

FIG. 25 the view according to arrow XXV in FIG. 24;

FIG. 26 a partially broken side view against the nebulizer, showing a gear and a sleeve part of the counter;

FIG. 27 individual perspective view of the sleeve part of the counter with the associated lower housing inner part;

FIG. 28 individual perspective view of a transmission part that can be assigned to the counter;

FIG. 29 a cross-section according to line XXIX-XXIX in FIG. 26;

FIG. 30 the section according to line XXX-XXX in FIG. 26;

FIG. 31 a sectional view according to FIG. 29, concerning an alternative design;

FIG. 32 a sectional view according to FIG. 30, concerning an alternative design;

FIG. 33 in Perspective View, the Nebulizer in a Ready Position to Receive the container;

FIG. 34 shows the base area of the container with the container connection part from an explosion perspective;

FIG. 35 in a simplified illustration, shown without the lower housing part, a blocking position of the sleeve part as well as a position before inserting a new container provided with a container connection part;

FIG. 36 a continuation of FIG. 35, an intermediate position in the course of inserting the container into the housing of the nebulizer;

FIG. 37 a perspective rear view of the illustration in FIG. 34;

FIG. 38 the container connection part in a further explosion perspective illustration;

FIG. 39 the nebulizer in a side view;

FIG. 40 the nebulizer partially cut along the line XL-XL in FIG. 39, concerning the first embodiment of the container connection part;

FIG. 41 a cut along line XLI-XLI in FIG. 40;

FIG. 42 the area XLII in FIG. 40, but an intermediate position in the course of inserting a new container into the housing;

FIG. 43 an enlarged perspective view of a cover of the container connection part;

FIG. 44 an individual perspective view of an insertion locking part;

FIG. 45 a representation corresponding to FIG. 42, with further displacement of the container together with the container connection part in the direction of an insertion position;

FIG. 46 a continuation of FIG. 45, with further displacement;

FIG. 47 an another illustration essentially corresponding to FIG. 42, but in the course of a removal displacement of the container;

FIG. 48 a continuation of FIG. 47;

FIG. 49 a continuation of FIG. 48;

FIG. 50 another illustration essentially corresponding to FIG. 42, but in the course of an attempt to insert an already used container into the housing;

FIG. 51 a representation of a container connection part in a second embodiment substantially corresponding to FIG. 37;

FIG. 52 an individual perspective view of the insertion locking part of the second embodiment according to FIG. 51;

FIG. 53 a perspective rear view against the insertion locking part of FIG. 52;

FIG. 54 a representation corresponding to FIG. 42, but concerning the embodiment according to FIG. 51;

FIG. 55 a continuation of FIG. 54, in the course of a removal displacement of the container connection part;

FIG. 56 is a continuation of FIG. 55;

FIG. 57 a perspective sectional view of the container connection part;

FIG. 58 a perspective cutaway view of the insertion locking part;

FIG. 59 an another subsequent illustration, concerning the activated position of the insertion locking part;

FIG. 60 a continuation of FIG. 59, concerning the temporary displacement of the insertion locking part into a non-activated position;

FIG. 61 a representation corresponding to FIG. 50, but concerning the second embodiment;

FIG. 62 an another illustration, substantially corresponding to FIG. 37, of a container connection part in a third embodiment;

FIG. 63 an individual perspective view of the insertion locking part of the third embodiment according to FIG. 62;

FIG. 64 in a fourth embodiment, a container connector in a representation substantially as shown in FIG. 37;

FIG. 65 individual perspective view of the insertion locking part of the fourth embodiment;

FIG. 66 an another illustration, substantially corresponding to FIG. 37, of a container connector in a fifth embodiment;

FIG. 67 the insertion locking part of the fifth embodiment according to FIG. 66 in a single perspective view.

### DESCRIPTION OF THE EMBODIMENTS

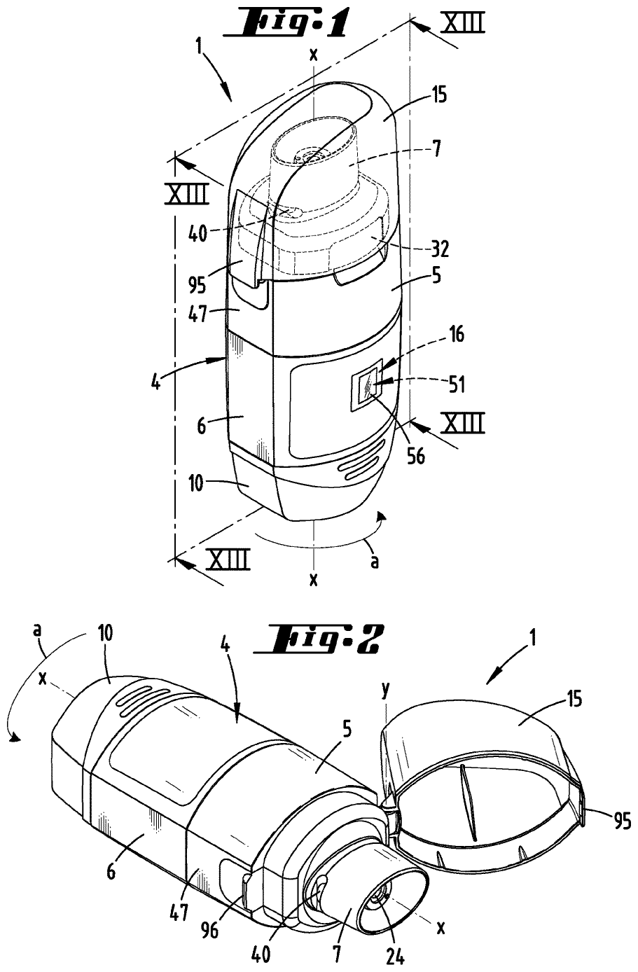

Shown and described, first with reference to the illustrations in FIGS. 1 to 3, is a nebulizer 1 for spray dispensing a fluid 3 stored in a replaceable container 2.

The nebulizer 1 is preferably designed as a portable inhaler, further preferably having an elongated, oval cross-section with preferably flattened narrow sides shape, with a length of about 80 to 200 mm, considered in the direction of a longitudinal housing axis x, further preferably about 100 to 150 mm, and a width of about 20 to 60 mm, further preferably about 25 to 50 mm, considered transversely thereto. Accordingly, the nebulizer 1 can be guided with one hand, in particular when carrying out an inhalation process.

The fluid 3 is preferably a therapeutic or pharmaceutical product to be inhaled by the user in the course of an inhalation process. Preferably, such inhalation is performed without propellant gas. Rather, the fluid 3 is preferably sucked from the container 2 into a pressure chamber 23 and, as a result of pressurization, expelled in an atomizing manner via a mouthpiece 7. The atomization processes carried out are preferably counted using a counter 16 arranged on the housing side.

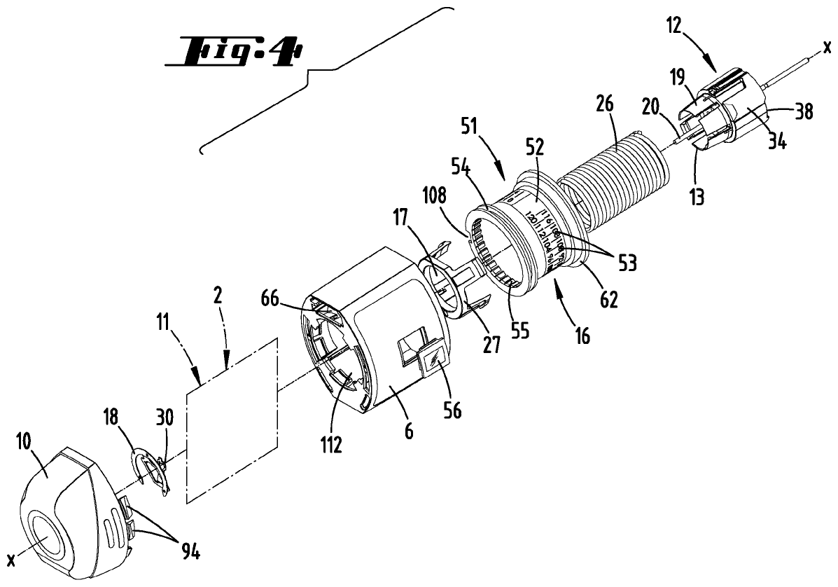

The nebulizer 1 has a housing 4 which is essentially divided into an upper housing part 5 and a lower housing part 6 (cf. essentially FIGS. 4 and 5), which housing parts 5 and 6 can be rotated relative to one another about the longitudinal housing axis x to a stop-limited extent. For the description of the drawings, the upper housing part 5 with the mouthpiece 7 formed thereon is defined as a stationary housing part, relative to which the lower housing part 6 can be rotated (see direction of rotation a in FIG. 1).

Viewed in the circumferential direction, the housing 4 of the nebulizer 1 can be provided with two opposing rounded sides and with two flat sides—offset by 90 degrees with respect to the housing longitudinal axis x—and arranged opposite one another. In this case, one flat side 47 of the upper housing part 5 covers an area accommodating a locking device S for the container 2 and/or for the counter 16. A viewing window 56 for the counter 16 can be provided in the region of a rounded side of the lower housing part 6.

The container 2 is replaceably received or receivable in the housing 4 of the nebulizer 1, in particular in a receiving space 112 in the lower housing part 6. In this case, the container 2 has a substantially circular-cylindrical or cartridge-like shape, wherein a collapsible, collapsible bag 111, in which the fluid 3 is held, is preferably accommodated in an outer rigid casing, substantially comprising a circumferential container wall 8 and a container base 9.

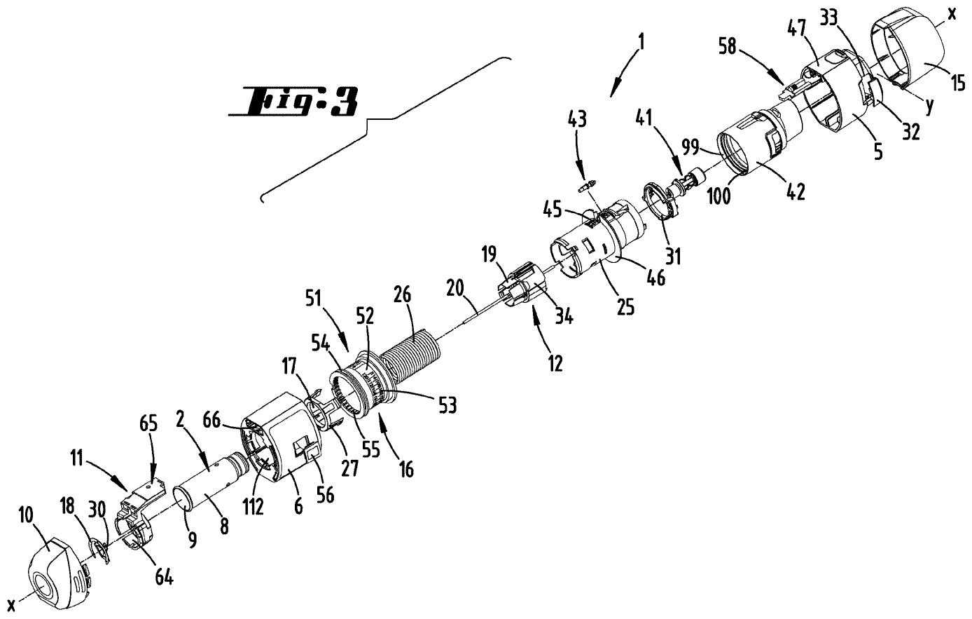

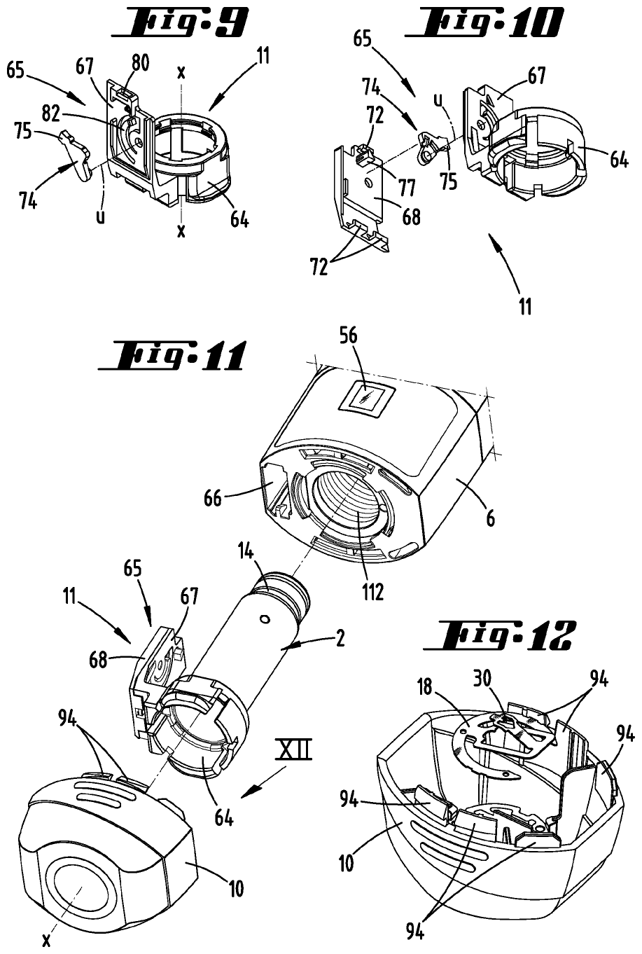

In the usual position of use, as shown for example in FIGS. 1 and 2, a closure cap 10 is provided on the lower housing part 6 in the area of the end facing away from the upper housing part 5. This can be snap-fitted to the lower housing part 6, for example by means of latching projections 94 of spring-out design. After removal of the closure cap 10 by overcoming the detent position according to FIG. 11, which removal can preferably be carried out without tools by the user if required, the container 2, preferably together with a container connection part 11, can be inserted from below into the housing 4, in particular into the lower housing part 6 or into the receiving space 112, or an inserted container 2 together with the container connection part 11 can be removed from the housing 4.

In the area of an end region facing the mouthpiece 7, the container 2 is snap-fitted to a suction tube part 12 guided linearly along the longitudinal axis x of the housing and non-rotatably relative to the housing inner part 25 in the lower housing part 6 or a lower housing inner part 25 connected non-rotatably to the housing part 6. Spring-loaded, radially inwardly directed latching projections 13 of the suction tube part 12 engage in a circumferential locking groove 14 of the container 2 in the area of its container neck (see FIGS. 14 to 16).

In a non-use position, as shown for example in FIG. 1, the mouthpiece 7 is preferably covered by a cover cap 15. The cover cap 15 can be pivotally displaceably mounted on the housing 4, here in particular on the upper housing part 5. The geometric pivot axis y enabling this pivotal displacement may extend substantially perpendicularly to the longitudinal axis x of the housing. In the cover cap closed position shown, for example, in FIG. 1, the cover cap 15 can engage behind a counter latching section 96 provided on the housing side by means of a latching section 95 to secure the closed position.

The pivot axis y and the latching and counter latching sections 95, 96 enabling latching of the cover cap 15 can, as is preferred, be provided associated with the flat sides of the housing parts 5 and 6.

The suction tube part 12 serves in particular for dispensing and atomizing the fluid 3, preferably in a defined metered quantity. For this purpose, the suction tube part 12 has, in addition to the holder 19 for the container 2, which has the latching projections 13, a suction tube 20 which, when the container 2 is latched to the holder 19, dips into the interior of the container, in particular into the interior of the bag 111 accommodated in the container 2, while piercing a passage opening 21 of the container 2.

The end of the suction pipe 20 facing away from the container 2 and pointing upwards in the illustrations can be provided at the end with a non-return valve 22, this end region of the suction tube 20 being inserted in the pressure chamber 23 formed in a nozzle body 41 so as to be linearly displaceable along the longitudinal axis x of the housing. Viewed in the direction of extension of the longitudinal axis x of the housing, an ejection nozzle 24 can be formed at the end of the pressure chamber 23 associated with the mouthpiece 7.

The suction tube part 12 can be spring-loaded in the direction of the mouthpiece 7, preferably with a limited stop. For this purpose, as is also preferred, a compression spring 26, for example in the form of a cylinder compression spring, can be provided, which preferably extends concentrically to the longitudinal axis x of the housing. The compression spring 26 is supported in the region of one end on the underside of the suction tube part 12, surrounding the holder 19, and in the region of the opposite and, according to the illustrations, downwardly pointing end on a base part 27 latched to the housing inner part 25. This base part 27 is provided with a central opening 17 through which the container 2 can project downwardly into the region of the closure cap 10 or through which opening 17 the container 2 can be inserted for latching to the holder 19.

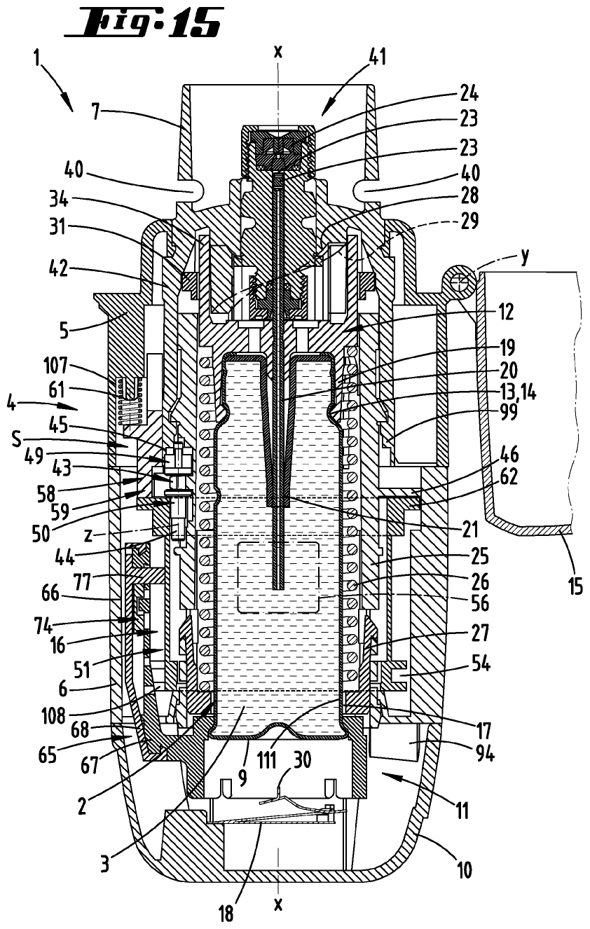

In preparation for an atomization or inhalation process, the compression spring 26 is tensioned, i.e. its axial extension is compressed. For this purpose, the upper housing part 5 is rotated relative to the lower housing part 6 about the longitudinal housing axis x, preferably by an angle of rotation of about 180 degrees. In the process, a spiral inclined surface 28 formed on the underside of the upper housing part 5, facing away from the mouthpiece 7, interacts with a facing counter inclined surface 29 of the suction tube part 12, which likewise slopes or rises in the circumferential direction, so that in the course of the rotation of the upper housing part 5 relative to the stationary lower housing part 6, a linear displacement of the suction tube part 12 with the container 2 fixed thereto is achieved along the housing longitudinal axis x with reference to the representation in FIG. 16 downwardly in the direction of the base part 27 against the restoring force of the compression spring 26 (compare FIG. 16). In the course of this linear displacement, fluid 3 is simultaneously sucked or conveyed from the container 2 via the suction tube 20 into the pressure chamber 23, which successively enlarges in the course of the displacement of the suction pipe tube 12.

For pressure equalization in the container 2, the container base 9 is pierced, in particular in the course of initial use of the container 2, during the downward displacement described above for clamping the system, for which purpose a piercing needle 30 is provided on the base side of the closure cap 10, preferably spring-supported in the direction of the longitudinal axis x of the housing (cf. FIGS. 12 and 16). The piercing needle 30 may, as shown, be part of a spring plate member 18 retained and formed in the closure cap 10 so that the piercing needle 30 is spring biased towards the container base 9.

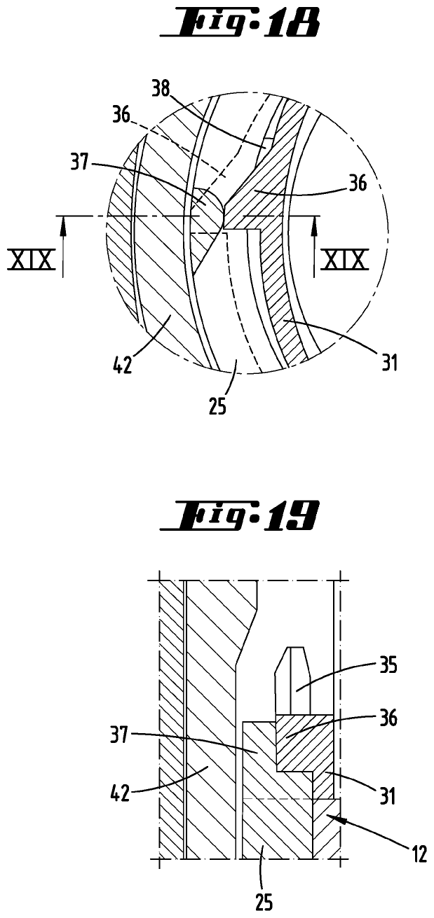

The tensioned position is initially secured. This is done in particular by a trigger ring 31 arranged in the upper housing part 5, which can be acted upon from the outside by the user via a trigger button 32. The trigger button 32 is ex-posed in a window 33 of the upper housing part 5 (compare FIGS. 5, 21 and 22).

In a non-use position of the nebulizer 1, the trigger button 32 is covered by the cover cap 15 and held in a protected concealed position.

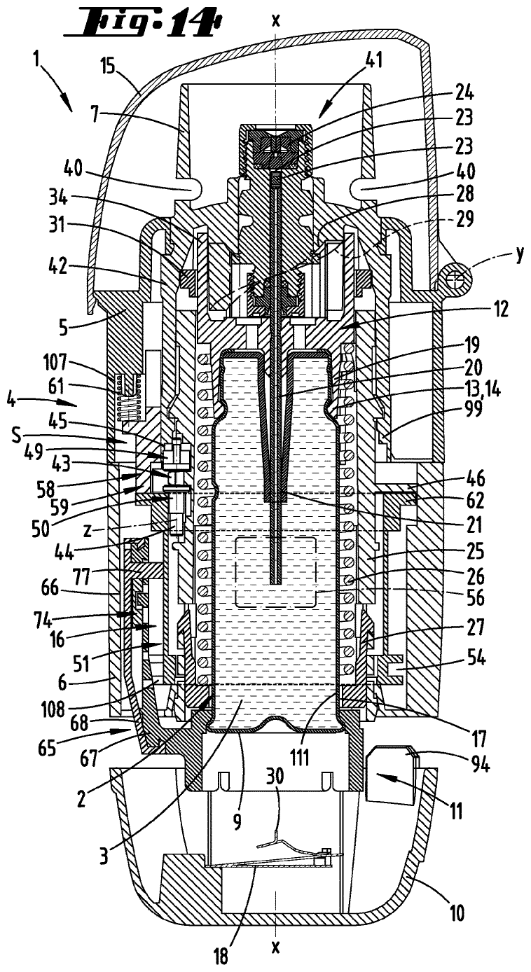

The trigger ring 31 initially and substantially surrounds a wall 34 of the suction tube part 12 surrounding the area of the counter inclined surface 29, with an increase in the thickness d of the ring measured in the radial direction viewed from both sides in the circumferential direction from the area of the trigger button 32 (cf. FIG. 17). In the region essentially diametrically opposite the trigger button 32, the trigger ring 31 has a receptacle 35 for a housing-side rib which opens upwards in a U-shape with respect to an orientation of the nebulizer 1 as shown in FIG. 15, thereby ensuring a rotationally secure retention of the trigger ring 31 as a whole in the upper housing part 5.

With reference to the illustrations, the receptacle 35 is formed on the top side of the trigger ring 31. A control projection 36 is formed on the underside of the trigger ring 31 approximately overlapping in the direction of the longitudinal axis x of the housing. During preparation of the nebulizer 1 by relative rotation of the housing parts 5 and 6 in the circumferential direction, this projection interacts with a counter-control projection 37 of the lower housing inner part 25, which rotates in the course of the rotation relative to the stationary trigger ring 31, in such a way that loading of the trigger ring 31 in the region of the control projection 36 in a radially inward direction is achieved via inclined surfaces of the projections sliding against one another.

Due to the relative rotation of the housing parts 5 and 6 with respect to each other, a relative rotation of the suction pipe tube 12 with respect to the fixed trigger ring 31 is also achieved in this position, so that the control projection 36 can enter radially inwardly into a recess 38 formed on the end face of the wall 34 of the suction pipe tube 12.

If necessary, with overrunning of the counter-control projection 37, the control projection 36 can partially engage in the recess 38 in order to lock the clamped position, thereby supporting itself on a shoulder 60 of the suction pipe tube 12 which delimits the recess 38 in the circumferential direction, as well as, if necessary, further or alternatively thereto, bearing with a radial shoulder of the projection spaced in the circumferential direction of the release ring 31 from the inclined surface of the control projection 36 against an associated radial shoulder of the counter-control projection 37 which can be reached after overrunning (compare FIGS. 16 to 19).

This firstly provides a safeguard to prevent reverse rotation and/or further rotation of the lower housing part 6 relative to the upper housing part 5 in the clamped position.

In the clamping direction, the ring-side control projection 36 can be overrun by the counter-control projection 37.

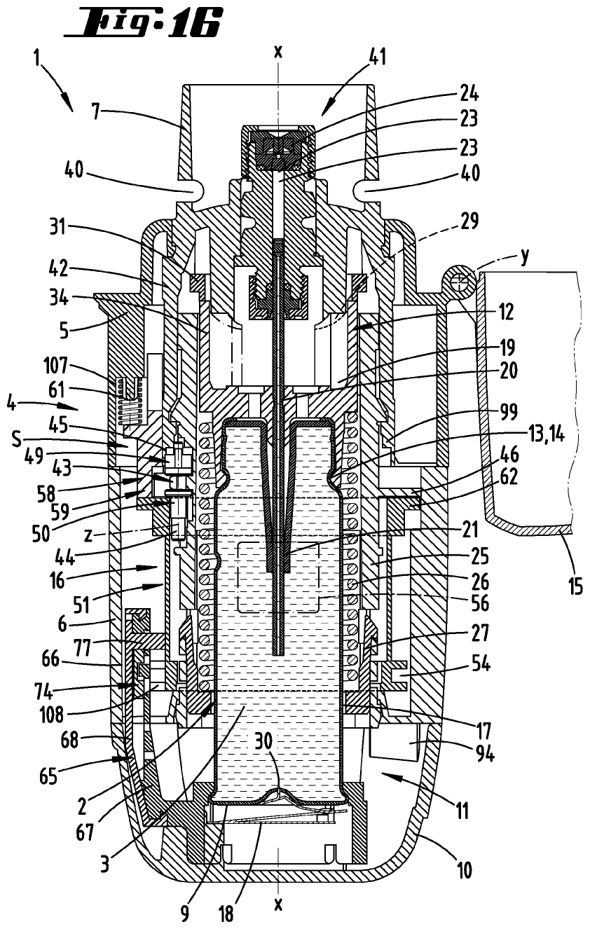

In addition, the tension of the release ring 31 when the suction pipe tube 12 is displaced downward, possibly supported by the radial displacement of the control projection 36 by the counter-control projection 37, may result from the design of the trigger ring 31 with a different thickness d in the circumferential direction, in particular in this region diametrically opposite the trigger button 32 having the control projection 36, an inward displacement of the trigger ring 31—preferably together with the control projection 36—in a radially inward direction results, so that the relevant section of the trigger ring 31 lies in front of the facing end edge surface of the wall 34 of the suction pipe tube 12 in a locking manner in the axial direction or steps in front of the bottom of the recess 38 in a locking manner engaging in the recess 38 (see FIGS. 18 and 19).

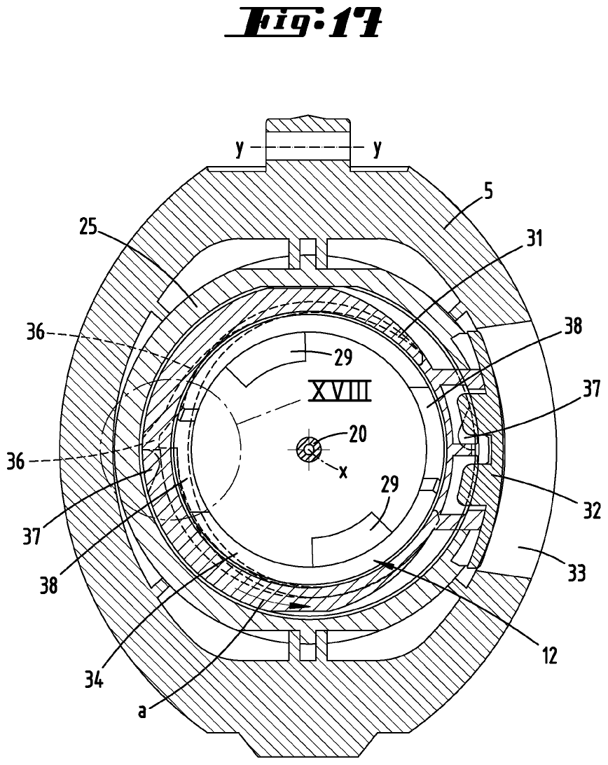

For inhalation, the user moves the cover cap 15 to a position exposing the mouthpiece 7 as shown in FIG. 2 and surrounds the mouthpiece 7 with the lips. With the subsequent unlocking of the compression spring 26, the fluid dose divided in the pressure chamber 23 is expelled via the ejection nozzle 24 (see FIG. 20). The user can inhale the emerging aerosol 39, whereby air can be sucked in via at least one ventilation opening 40 at the foot side of the mouthpiece 7.

Unlocking is effected by a pressure actuation of the trigger button 32 by the user, whereby the trigger ring 31 is loaded in such a way that its area essentially opposite the trigger button 32, in particular having the control projection 36, is displaced back radially outward again, which leads to a release of the suction pipe tube 12. Accordingly, this can spring back into the starting position as a result of the release of the restoring force of the compression spring 26, whereby the fluid is expelled from the pressure chamber 23 and via the ejection nozzle 24 via the now closed non-return valve 22, which serves quasi as a piston in the course of this process. This can result in a nozzle jet fan of the aerosol 39 as shown in FIG. 20, with an opening angle of about 30 to 150 degrees, further about 75 to 115 degrees.

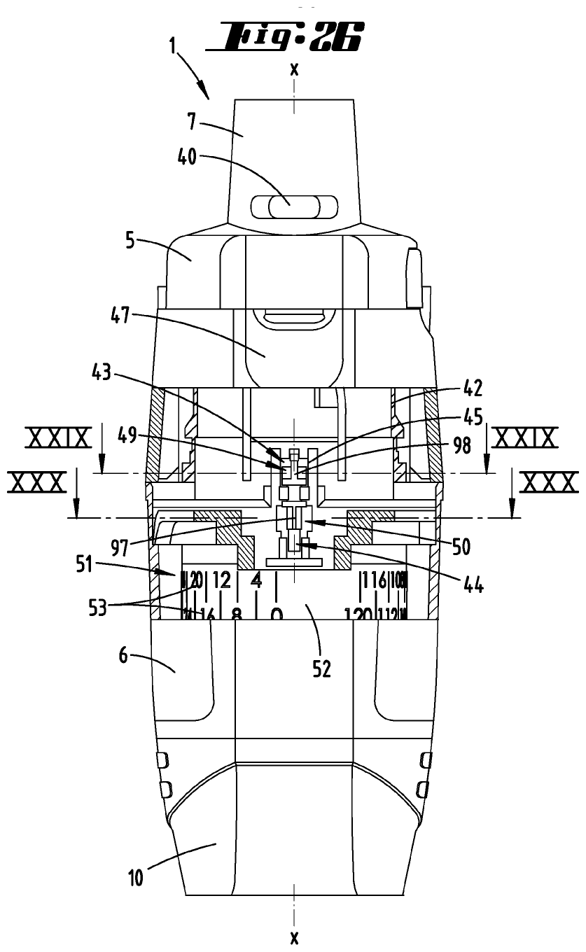

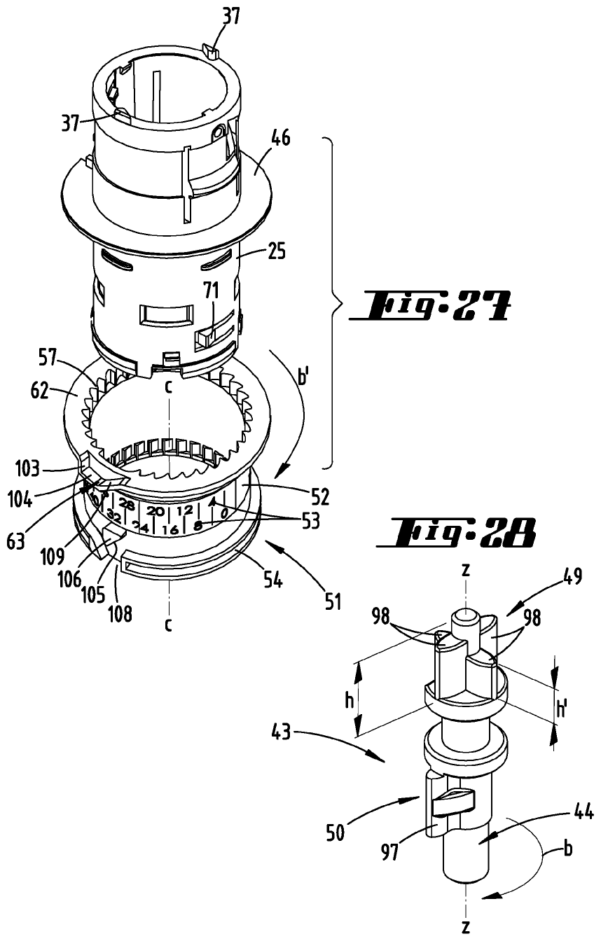

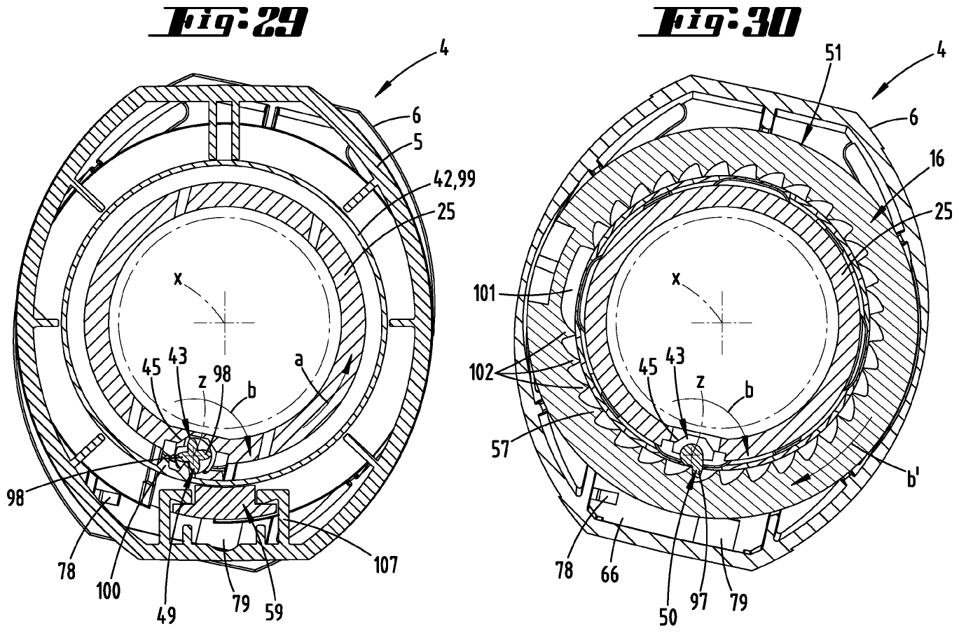

An atomization operation can be registered and counted using the counter 16 housed in the housing 4. The counting takes place with the rotation of the housing parts 5 and 6 relative to each other, that is, with the preparation of the nebulizer 1 for carrying out a fluid discharge. It is not necessary that each rotation of the housing parts 5 and 6 relative to each other causes an action on the counter 16 in the sense of a count (compare FIG. 32). For example, only every second (compare FIG. 31) or even every fourth rotation (compare FIGS. 29 and 30) can lead to an action on the counter 16 in the sense of a count.

Part of the counter 16 is a transmission part 43, which transmits the relative rotational displacement of the housing parts 5 and 6 to each other to a sleeve part 51 of the counter 16. This sleeve part 51 is provided with characters 53 applied to an outer surface 52, which represent the counts.

The transmission part 43 is designed in the manner of a transmission shaft 44, with an axis of rotation z which runs in the same direction as the longitudinal housing axis x. The transmission part 43 is further designed and arranged in such a way that it cooperates directly with the upper housing part 5, in particular with an upper housing inner part 42 surrounded by the upper housing part 5, as well as with the lower housing part 6, in particular with the lower housing inner part 25, and also directly with the sleeve part 51 of the counter 16.

For this purpose, the transmission part 43 is rotatably held in a niche 45 formed on the outside of the wall of the lower housing inner part 25 and open radially outwards, both the niche 45 and the transmission part 43 in the position of use passing through a transverse plane running transversely to the housing longitudinal axis x, in which transverse plane a collar-like circumferential first support flange 46 is formed at the same time on the outside of the wall of the lower housing inner part 25.

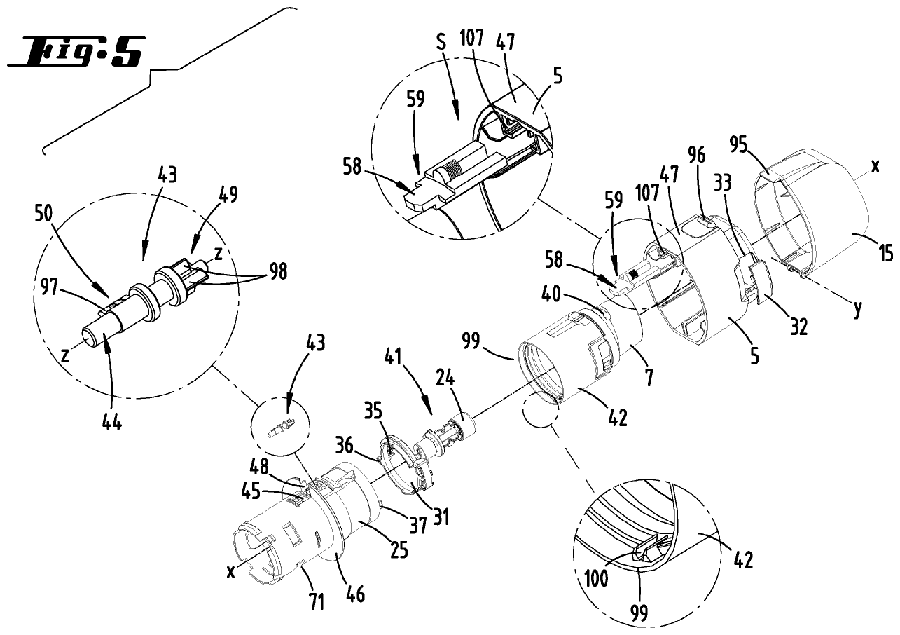

Associated with the niche 45, the first support flange 46 is provided with an open-edge passage opening 48 (compare FIG. 6).

The transmission shaft 44 has two areas spaced apart from one another in the direction of its axis of rotation z, with a first toothed formation 49 and a second toothed formation 50. Thus, with reference to an orientation of the nebulizer 1 as shown in FIG. 26, the second toothed formation 50 can be formed to be associated with the lower area of the transmission shaft 44, below the support flange 46, and the first toothed formation 49 can be formed correspondingly to the upper area, above the support flange 46.

For example, according to the representation of the transmission part 43 in FIG. 28, the second toothed formation 50 can be formed by a driving finger 97 projecting essentially radially outward with respect to the axis of rotation z, for cooperation with a circumferential toothing 57 formed on the inside of the wall of the sleeve part 51. Accordingly, this results here in a toothing engagement directed in the transverse direction to a sleeve axis c (compare FIG. 30).

The first toothed formation 49 may be formed by four counting fingers 98 evenly distributed in the circumferential direction on the transmission shaft 44 (compare FIG. 28). These may, as is preferred, have equal radial lengths. With regard to the heights h and h′ considered in the axial direction, the arrangement can be further selected in such a way that a pair of diametrically opposed counting fingers 98 have a height h that is greater by a factor of 1.1 to 1.5, for example, than the other pair of opposed counting fingers 98.

The upper housing inner part 42, which is non-rotatably connected to the upper housing part 5, encloses the lower housing inner part 25 with a free wall end section 99 pointing in the direction of the sleeve part 51 in a region of the first toothed formation 49 of the transmission part 43 held in the lower housing inner part 25. On the inside of this wall end section 99, correspondingly facing the lower housing inner part 25, a driver 100 of approximately U-shaped design is formed in a plane transverse to the housing longitudinal axis x (compare enlarged detailed representation in FIG. 5), for cooperation with the first toothed formation 49.

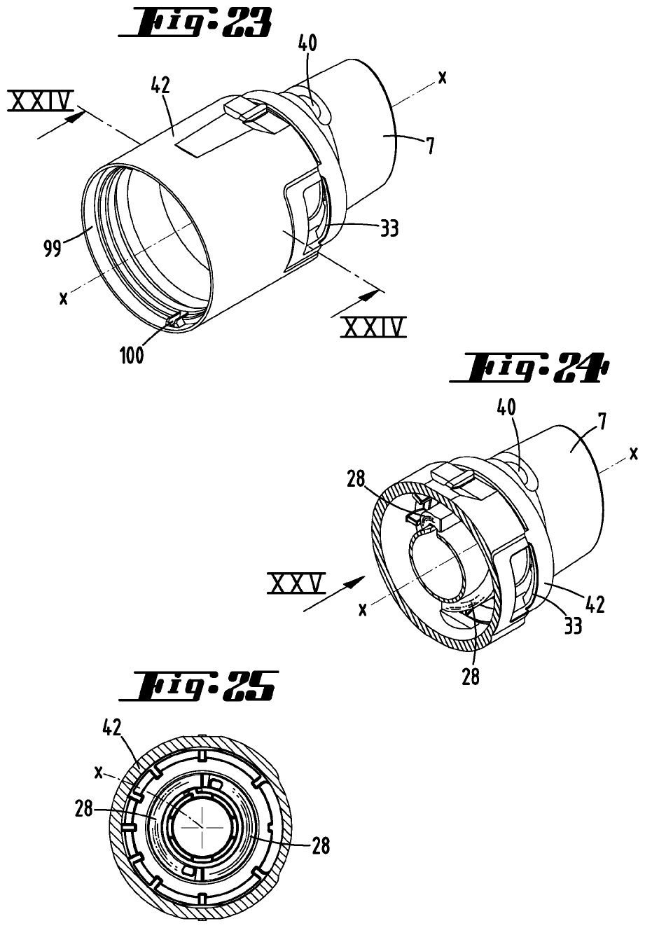

With reference to the illustrations, the first toothing 57 of the sleeve part 51 cooperating with the second toothed formation 50 is formed in an upper edge region of the sleeve part 51 on the sleeve inner side. At an axial distance there-from in the region of a lower edge, a second circumferential toothing 55 is preferably likewise formed on the inside of the sleeve, for cooperation with a spring-out return locking finger 71 formed on the lower inner housing part 25 (compare FIG. 27).

Two circumferentially adjacent tooths 102 of the first toothing 57 may form an acute angle of about 8 to 12 degrees with respect to each other, for example, each with respect to a radially inwardly facing tooth tip, further for example, about 10.5 degrees. Thus, a preferred number of 30 to 35 teeth, more preferably 32 teeth, may further result in a toothed region extending in the circumferential direction over a total angle of about 330 to 340 degrees, more preferably about 336 degrees.

Over the remaining circumferential portion of, for example, 20 to 30 degrees, further for example 24 degrees, there is a tooth gap 101 which provides a free run for the driving finger 97 of the transmission part 43, particularly in the course of a container change described in more detail below.

Further, a circumferential guide groove 54 may be provided associated with the outer surface 52 on the sleeve part 51 which, in conjunction with a portion of the lower housing member 6 engaging this guide groove 54, provides rotational retention of the sleeve part 51 within the lower housing member 6 as a whole.

The characters 53 applied to the outer surface 52 are visible to the user from the outside through a transparent viewing window 56 provided in the lower housing part 6, in particular the character 53 representing the current filling status of the inserted container 2.

With a rotation of the lower housing part 6 in the direction of rotation a relative to the fixed upper housing part 5 for the preparation of an atomization process, a rotation of the toothed formation 49 as a whole about its axis of rotation z (direction of rotation b) by preferably 180 degrees can be effected as a result of the action of the driver 100 provided in the upper housing inner part 42 on the first toothed formation 49 of the transmission part 43 (compare FIG. 29).

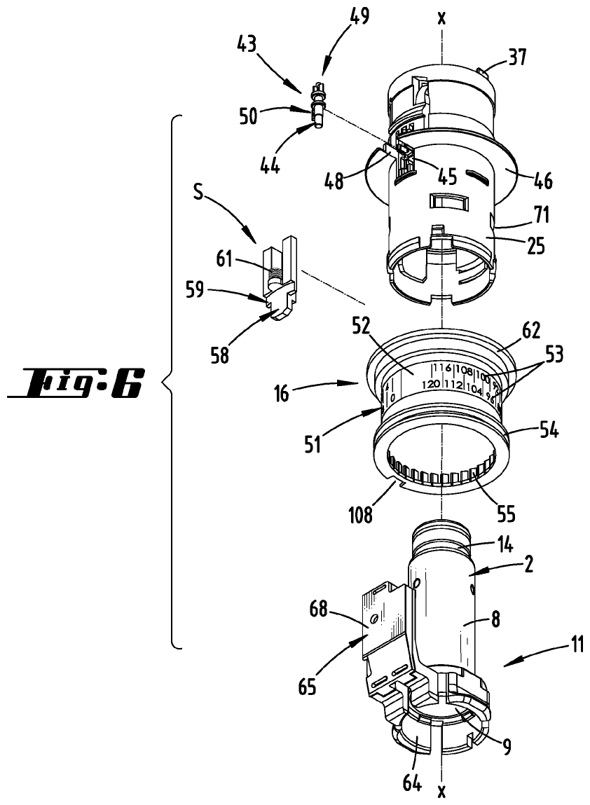

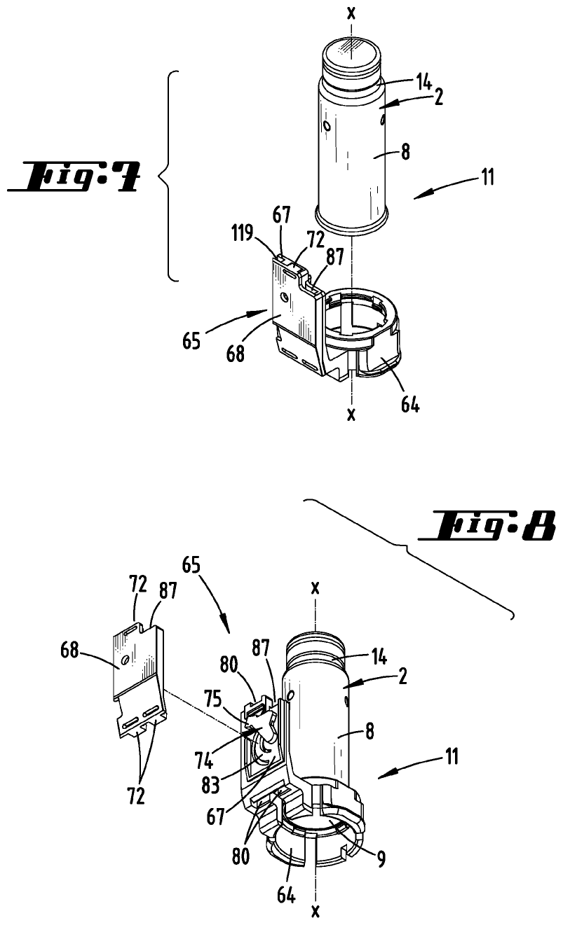

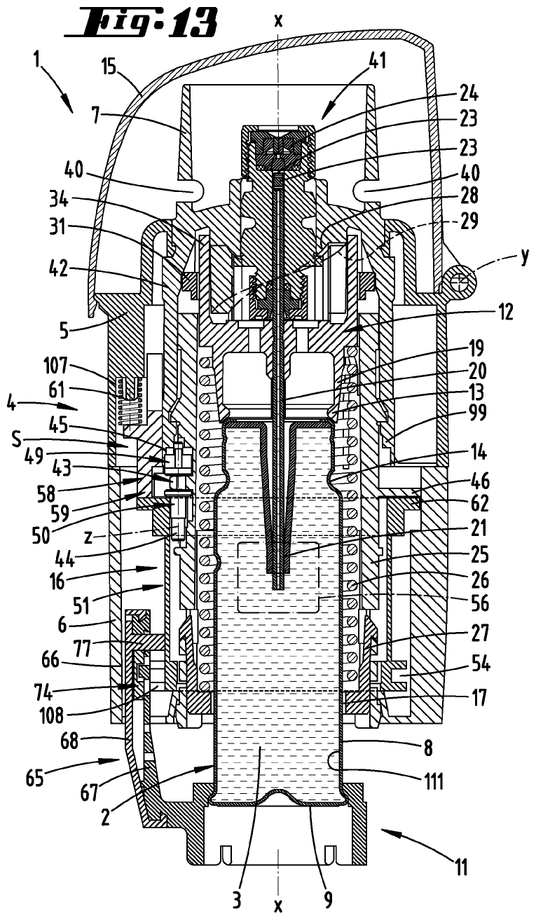

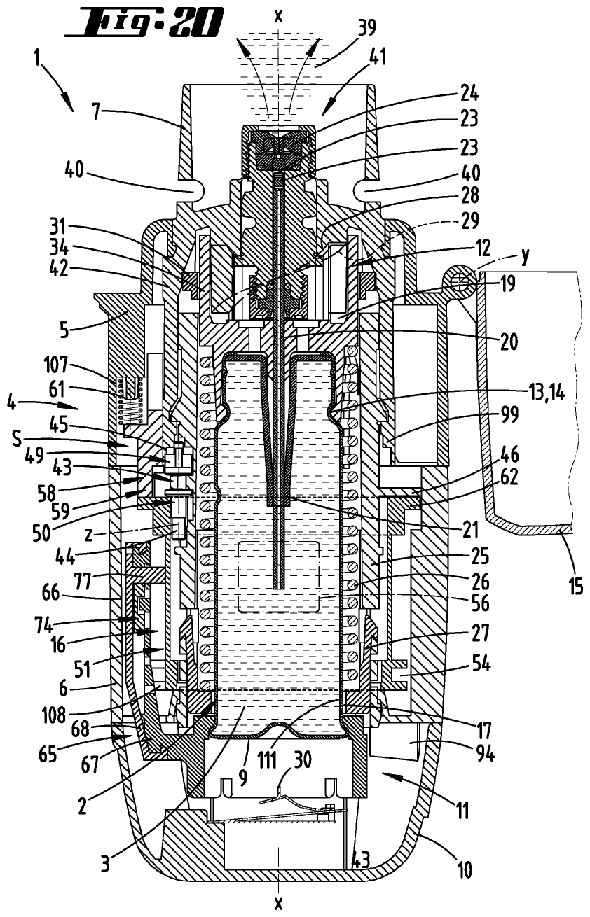

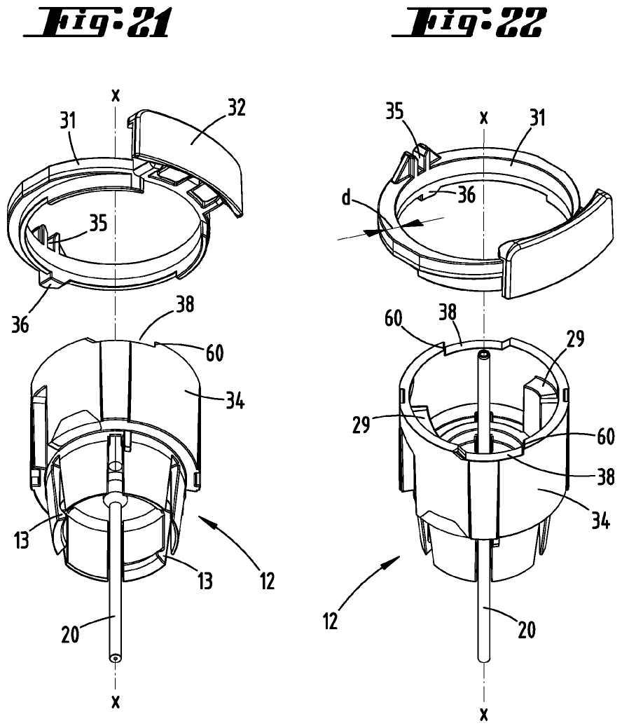

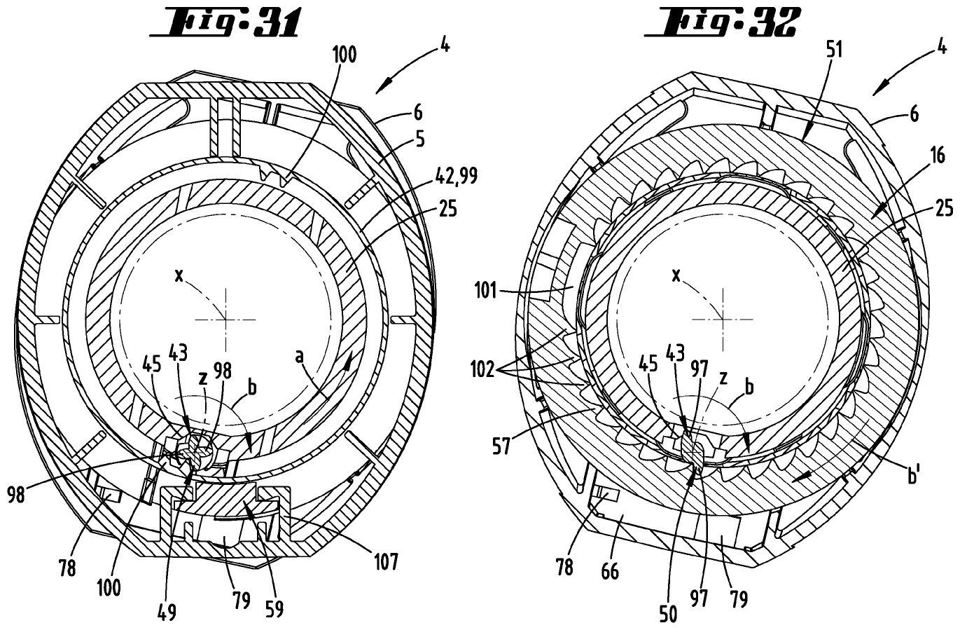

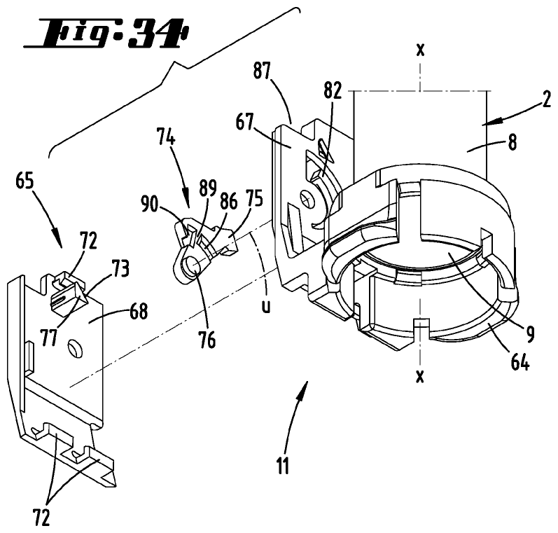

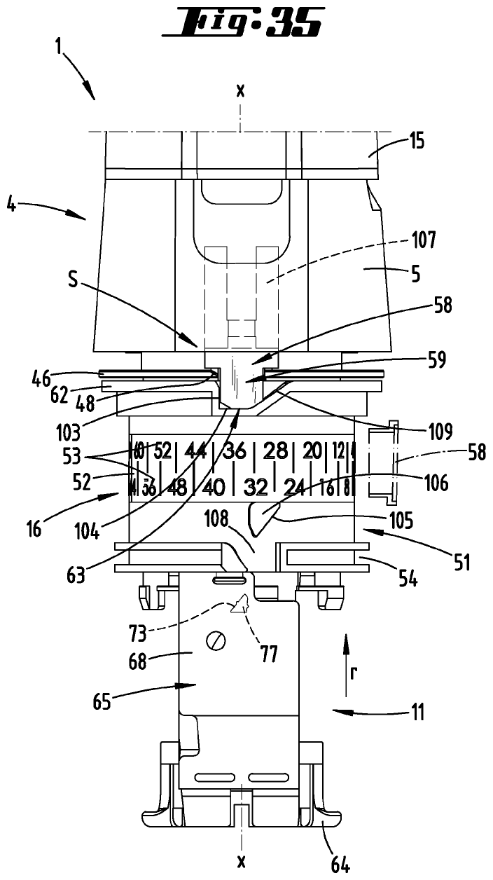

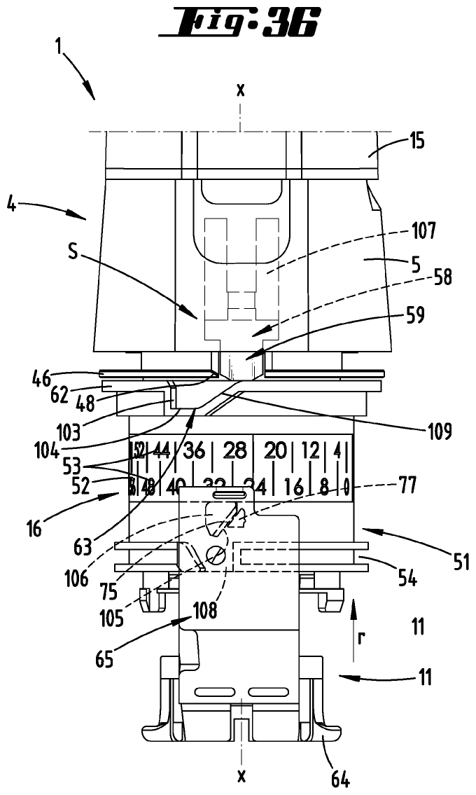

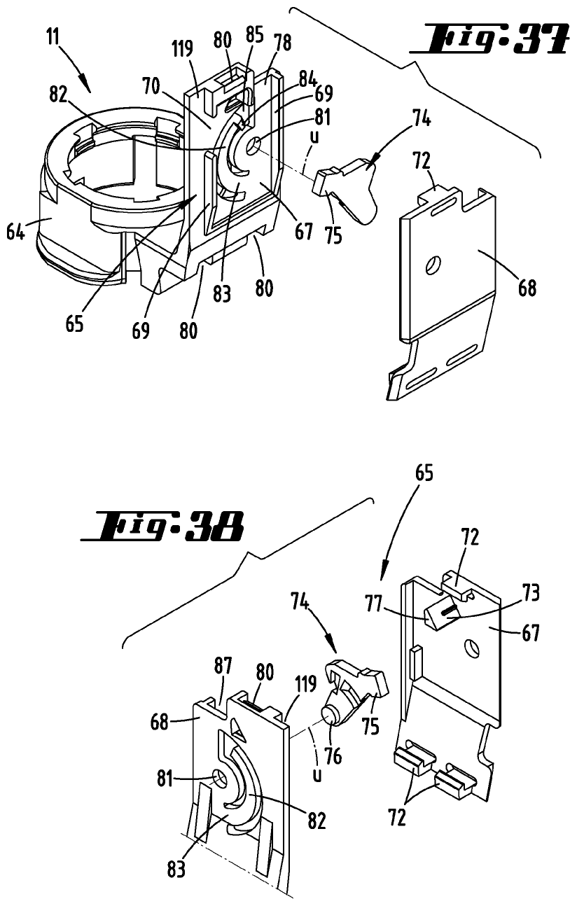

In the first embodiment shown in particular in FIGS. 1 to 30 and 33 to 50, only one driver 100 is provided (compare FIG. 29). Accordingly, a rotation of the housing parts 5 and 6 relative to each other by 180 degrees per actuation only results in an action on the first toothed formation 49 of the transmission part 43 with every second preparatory actuation of the nebulizer 1 and thus an associated total rotation of the lower housing inner part 25 holding the sleeve part 51 with the first toothing 57 by 360 degrees.

In conjunction with the single driving finger 97 provided in this first embodiment, a rotation of the second toothed formation 50 with the driving finger 97 in the direction of rotation b by 180 degrees results every second time an atomization preparation position is reached, due to the action on the transmission part 43 via the driver 100. The attainment of every fourth atomization preparation position causes a rotating action on the sleeve part 51 in the direction of rotation b′ via the driving finger 97, which is then rotated through 360 degrees in the direction of rotation b overall. The driving finger 97 moves the sleeve part 51 further by one tooth 102, correspondingly preferably the sleeve part 51 over an angle of 10.5 degrees.

In the embodiment shown, the characters 53 are preferably counted by a count value four starting from a maximum count value (for example 120) towards a minimum count value (for example zero).

In order to achieve, for example, a counting pulse on the sleeve part 51 when every second atomization preparation position is reached, with an overall substantially identical design of the nebulizer components, the upper inner housing part 42 can be provided with two diametrically opposed driving fingers 100 in accordance with the further embodiment shown in FIG. 31. Accordingly, each relative rotation of the housing parts with respect to one another results in a rotational action over 180 degrees in each case on the transmission part 43, so that further with arrangement of only one driving finger 97 a 360 degree rotation of the same is achieved for acting on the sleeve part 51 after two atomization preparation positions. For example, with the same number of tooths 102 and a respective angular step of about 10.5 degrees, it is possible in this case to count down by one count value two at a time, starting from a count maximum value of sixty.

According to a further embodiment shown in FIG. 32, the second toothed formation 50 can also be provided with two driving fingers 97, which are also diametrically opposed, so that each 180 degree rotation of the transmission part 43, i.e. each interaction of the first toothed formation 49 with a driver 100, causes a rotational driving of the sleeve part 51 in the direction of rotation b′ by one tooth 102 and thus a registration/counting.

In cooperation with an upper housing inner part 42 with only one driver 100 according to the first embodiment, an alternative design is thus achievable by means of which a counting pulse is transmitted to the sleeve part 51 upon reaching every second atomization preparation position.

If, as described with reference to the further embodiment shown in FIG. 31, two diametrically opposed drivers 100 are provided, then in conjunction with the double arrangement of driver fingers 97 a rotational entrainment of the sleeve part 51 by a count amount is achieved when each individual atomization preparation position is reached. With the same number of tooths 102 and a respective angular step of about 10.5 degrees, for example, it is possible in this case to count off by a count value of one in each case, starting from a count maximum value of thirty.

In an advantageous manner, the counter 16 and, via this, the nebulizer 1 are blocked by the locking device S in the sense of the rotatability of the housing parts relative to one another, in particular when a minimum count value (for example zero) is reached. Accordingly, the blockade does not permit further rotation of the lower housing part 6 relative to the upper housing part 5. The user is thus also made aware haptically of an emptied container 2 without having to look separately at the counter display. The nebulizer 1 cannot be used without further measures.

For this purpose, the locking device S preferably has a locking pawl 59 with a locking projection 58. The locking pawl 59 can, as also shown, be assigned to one of the flat sides 47 described above and held in a guide 107 formed on the inside of the wall of the upper housing part 5 so as to be displaceable in the direction of extension of the longitudinal axis x of the housing, preferably with a limited stop. In this case, the locking pawl 59 is further preferably spring-loaded via a spring 61, for example, as also shown, via a cylinder compression spring, in the direction of a detent position according to the downward illustrations, in the direction of the sleeve part 51 (cf. FIGS. 5 and 16).

In normal operation of the nebulizer 1, the locking pawl 59 is supported with its locking projection 58 on the tip side on a second support flange 62 surrounding the first toothing 57 of the sleeve part 51 or—possibly depending on the rotational position—on the first support flange 46 of the lower housing inner part 25. The support surface of the second support flange 62 extends transversely to the housing longitudinal axis x, preferably viewed along the housing longitudinal axis x substantially directly below the first support flange 46, offset in the plane in the direction of the base part 27. In addition, the second support flange 62 extends radially outwardly to the first toothing 57.

Viewed over the circumference, this second support flange 62 is also partially interrupted. Accordingly, there is a circumferential locking opening 63 of the sleeve part 51 in this area, into which locking opening 63 the locking projection 58 automatically enters in a locking manner due to the spring load described above, in particular when the minimum count value (for example zero) is reached.

In such a count-minimum position of the sleeve part 51, the passage opening 48 of the first support flange 46 formed on the lower housing inner part 25 and the locking opening 63 of the second support flange 62 formed on the sleeve part 51 lie in overlap-with respect to the direction of extension of the housing longitudinal axis x. Furthermore, both flange openings (passage opening 48 and locking opening 63) extend together in the direction of travel of the locking pawl 59. In this circumferential alignment of the flange openings with the locking projection 58, the locking pawl 59 can, spring-loaded by the latter, enter a locking position according to FIG. 35, in which the locking pawl 59 crosses the transverse plane given by the supporting flanges and, by bearing in the locking opening 63 against a locking shoulder 103 preferably extending perpendicularly to the second supporting flange 62, effects a rotational locking between the housing parts in the usual direction of rotation a.

In the blocking position, the locking protrusion 58 may seat on a bottom section 104 of the locking opening 63 in a stop-limited manner.

In the illustrated embodiment examples, as is also preferred, this locking of the counter 16 can be released exclusively by removing the container 2 from the housing 4. In particular, the container connection part 11, which is connected to the container 2 and can further preferably only be separated from the container 2 by destruction, serves this purpose.

The container connection part 11 can, as is also preferred, be a plastic injection-molded part, further in particular having a pot-like base section 64 with a pot-shaped depression in which the container 2 is received at the foot end, i.e. in the region of the end opposite the locking groove 14. A cantilever 65, which is aligned in the direction of the longitudinal axis x of the housing in the state of assignment and extends adjacent to the container 2 in the direction of the mouthpiece 7, is integrally formed on this base section 64. With insertion of a container 2 into the housing 4 with the closure cap 10 open, this cantilever 65 can be inserted into a guide receptacle 66 of the housing 4, in particular of the lower housing part 6, this guide receptacle 66 being assigned to the region of the upper housing part 5 having the flat side 47 with the locking pawl 59 guided on the inside. The guide receptacle 66 extends adjacent to the receiving space 112 for the container 2.

According to the illustrations, the cantilever 65 has two plate parts 67 and 68 which run essentially parallel to one another and are spaced apart from one another perpendicular to their extension. The spacing from one another is achieved by bridge 69 running essentially parallel to the longitudinal axis x of the housing. One bridge 69 leaves a passage opening 70 approximately in an upper third of its longitudinal extent (compare FIGS. 37, 42 and 43).

Between the bridges 69 and covered by the plate parts 67 and 68, an insertion locking part 74 is further arranged with an outwardly facing plate-shaped latch section 75.

The plate parts 67 and 68 can be latched together to form the cantilever 65 while being spaced apart via the bridges 69. For this purpose, for example, latching projections 72 formed on the plate part 68 can be used, which can cooperate with corresponding counter latching areas 80 of the plate part 67.

In addition, the cantilever 65 facing the container 2 has a control projection 77 with a control surface 73 extending obliquely with respect to an insertion direction r of the container connection part 11 equipped with the container 2.

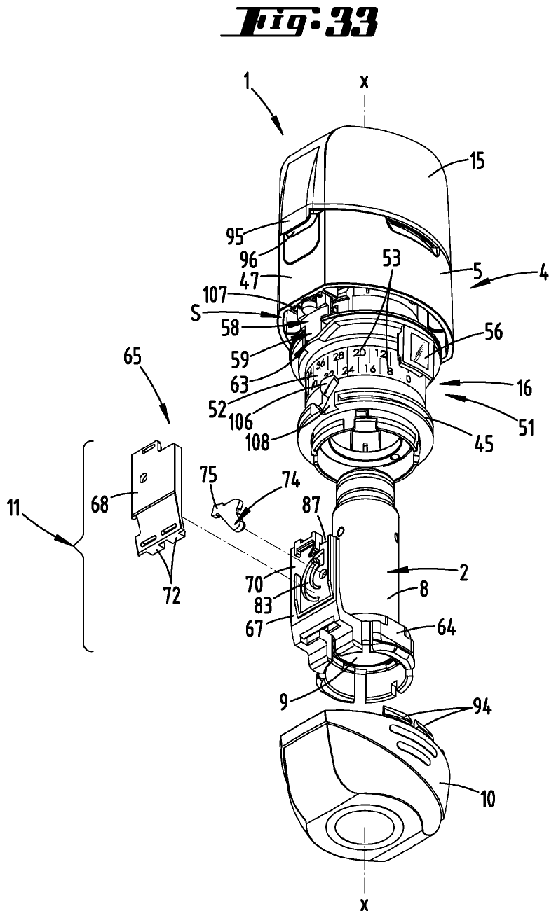

With the insertion of an unused container 2 with its container connection part 11 into the housing 4 (insertion direction r), the cantilever 65 is inserted into the guide receptacle 66. While passing through a circumferential interruption area 108 of the area of the sleeve part 51 forming the guide groove 54, the control surface 73 reaches a counter inclined surface 105 of a control section 106 provided on the outside of the wall of the sleeve part 51, which is steeply inclined in the same direction. As a result of this collision and the subsequent sliding of the inclined surfaces against each other in the course of the further insertion process, a rotation of the sleeve part 51 in the usual direction of rotation b′ is forced into its basic position (cf. FIGS. 35 and 36). The basic position is preferably the counting position in which the maximum counting value is displayed (e.g. 120, 60 or 30).

The formation of the tooth gap 101 in the region of the first toothing 57 of the sleeve part 51 results in free run for the driver finger 97, so that the adjustment of the sleeve part 51 into the basic position can be carried out without any intentional cancellation of a toothing engagement, for example by lifting or dis-placing a gear engagement part.

The angle of rotation achieved in this process corresponds essentially to the angular dimension over which the tooth gap 101 of the sleeve part 51 extends.

In the course of the thus achieved rotation of the sleeve part 51 in the course of the insertion process of a preferably new, unused container 2, the locking pawl 59 with its locking projection 58 is lifted against the force of the spring 61 from the blocking position according to FIG. 35, as a result of a control over a control slope 109 opposite the locking shoulder 103 of the locking opening 63.

The locking projection 58, which is preferably adapted with a phase, is lifted accordingly and finally rests on the facing surface of the second support flange 62 under spring action in the basic or initial position then reached as shown in FIG. 36.

In the final basic position, the latching projections 72 is supported by the area having the guide groove 54, which is radially expanded relative to the display area of the sleeve part 51.

From this basic position, during a usual rotation of the lower housing part 6 against the upper housing part 5, the locking projection 58 may be raised onto the first support flange 46 and slide thereon.

Preferably, only the insertion of a non-pre-used container 2 is provided. On the other hand, a pre-used container 2, including an emptied container 2, is prevented from being (re)inserted into the housing 4. The aforementioned insertion locking part 74 of the container connection part 11 is used for this purpose.

The drawings show various embodiments in this regard.

In the embodiment shown at, for example in FIGS. 33 to 50, the insertion locking part 74 is pivotably mounted on the cantilever 65. The geometric pivot axis u extends transversely to the insertion direction r and thus preferably transversely to the longitudinal axis x of the housing.

For pivotal locking, the insertion locking part 74 may include a pin 76 which is seated in a suitably adapted bearing opening 81 of the cantilever 65, for example in the region of the plate part 67 directly connected to the base section 64.

With reference to the direction of extension of the pivot axis u, a separate spring arm 82 is formed in the cantilever 65, for example in the plate part 67, in overlapping relationship with a pivot region of the insertion locking part 74. This further preferably extends essentially concentrically to the pivot axis u and, in the region of the end region facing away from the attachment area 83 of the spring arm 82 on the plate part 67, bears a latching projection 84 directed in the direction of the facing lower surface of the insertion-locking part 74.

In an imaginary further extension of the separate spring arm 82 beyond the latching projection 84, a free-cut evasion space 85 results in the plate part 67.

Facing the plate part 67 carrying the spring arm 82 with the latching projection 84, the insertion locking part 74 has a groove 86 aligned substantially concentrically with the pivot axis u, into which groove the latching projection 84 plunges before a first insertion of the container 2 into the housing 1 and thus holds the insertion locking part 74 in a basic position in which the latter is held substantially between the latching projection 84 and a bridge 69 opposite the passage opening 70 (compare, for example, FIG. 42).

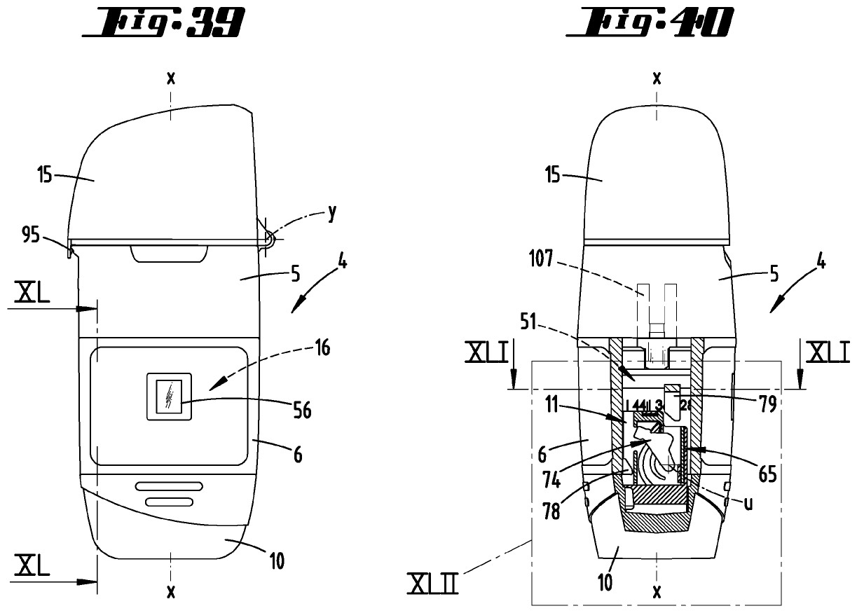

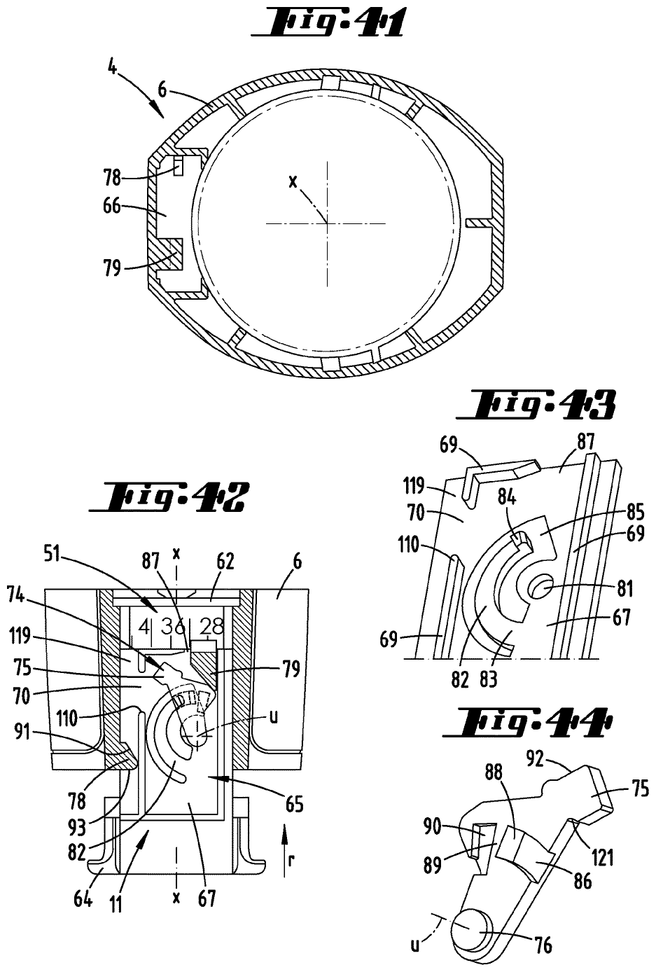

In the course of the insertion process of the container 2 with the cantilever 65 in the insertion direction r, a projection 79 on the housing side in the form of a control tongue emerges in the region of the guide receptacle 66 through a window 87 of the cantilever 65 and thus into the region between the plate parts 67 and 68 of the cantilever 65. The projection 79 loads the insertion locking part 74 as a result of being acted upon in the direction of rotation e (compare FIG. 45), wherein the latching projection 84 at the end of the groove 86 of the insertion locking part 74 can deflect under control via a control slope 88 in alignment with the pivot axis u to overrun a locking shoulder 89 and finally, in the course of further pivotal displacement of the insertion locking part 74, latches via the projection 79 into a latching receptacle 90 of the insertion locking part 74, which receptacle is separated from the groove 86 by the locking shoulder 89 (compare FIG. 46). The insertion locking part 74 is thus brought into an activated position and held in a latching manner in this position, in which position the latch section 75 projects through the passage opening 70 of the cantilever 65 and beyond an opening plane E of the passage opening 70 outwards into a passage gap 119 resulting between the plate parts 67 and 68.

To remove the container 2, the sealing cap 10 is first removed from the housing 4, after which, by grasping the container 2 preferably in the area of the container connection part 11, the container 2 can be pulled out of the housing 4 and out of the guide receptacle 66 with the cantilever 65 linearly along the longitudinal axis x of the housing in the removal direction r′.

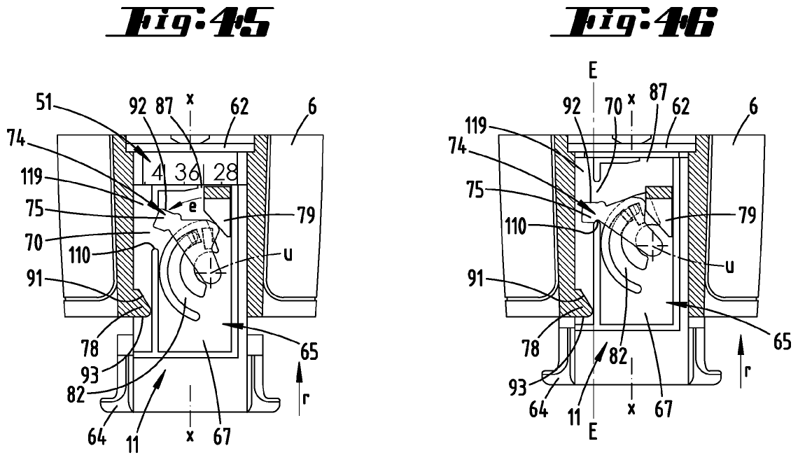

In the course of this linear displacement of the cantilever 65 in the removal direction r′, an obstacle projection 78 on the housing side enters the passage gap 119 and thus moves against the insertion locking part 74, in particular its latch section 75, which has been moved into the activated position (compare FIG. 47). As a result of the control of the latch section 75 via a control slope 91 of the obstacle projection 78, the insertion latch part 74 is loaded overall in the direction of a non-activated position (correspondingly in a deflection direction e′ against the direction of rotation e). The separate spring arm 82, which is latched to the insertion locking part 74 via the latching projection 84 engaging in the latching receptacle 90, can follow this deflecting movement in a resilient manner counter to the direction of rotation e. The free end of the spring arm 82, which has the latching projection 84, temporarily deflects into the evasion space 85 (compare FIG. 48).

When the housing-side obstacle projection 78 is overrun, the insertion locking part 74, in particular the latch section 75, is returned to the activated position as a result of the relaxed return movement of the spring arm 82, in which the latch section 75 projects beyond the opening plane E and into the passage gap 119 (compare FIG. 49).

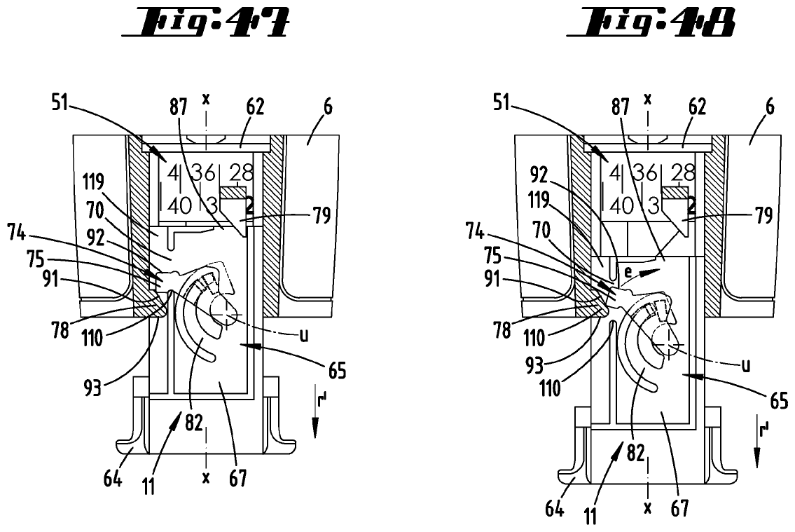

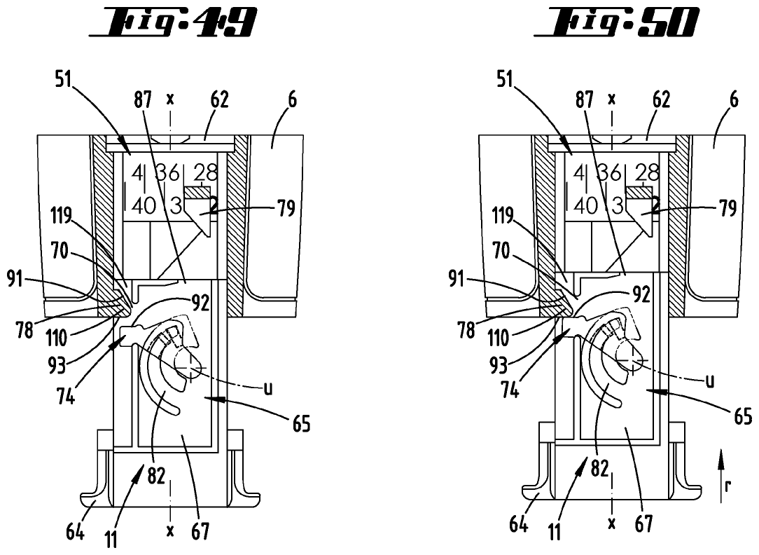

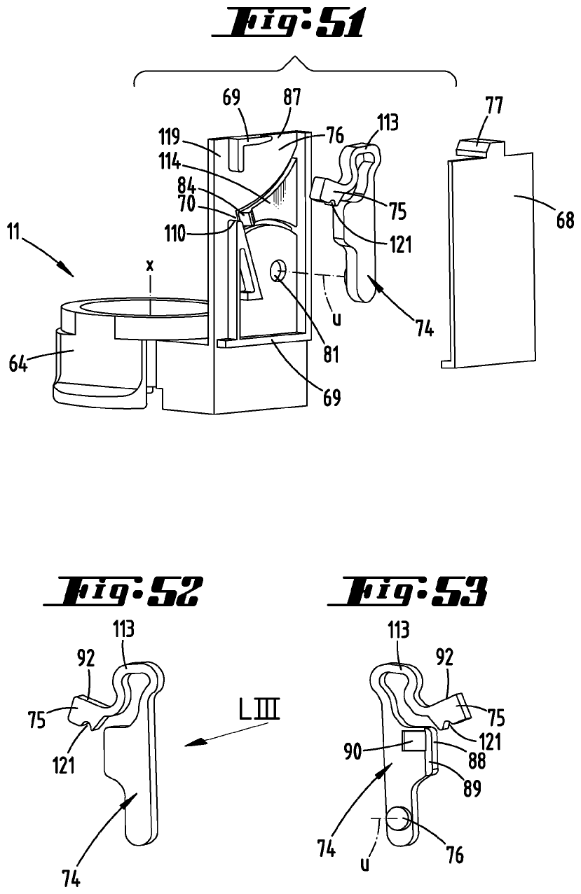

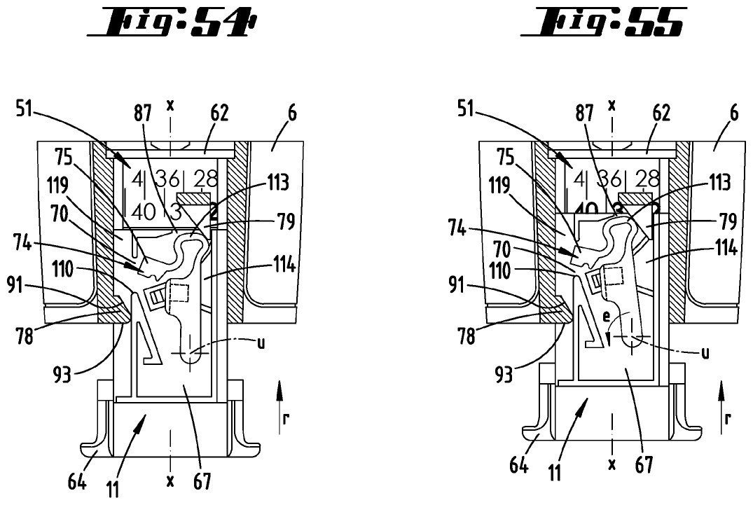

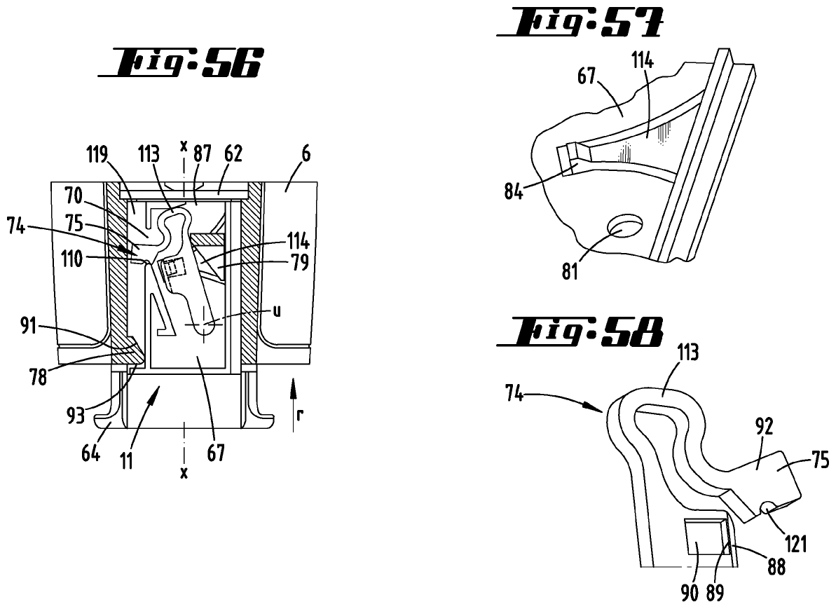

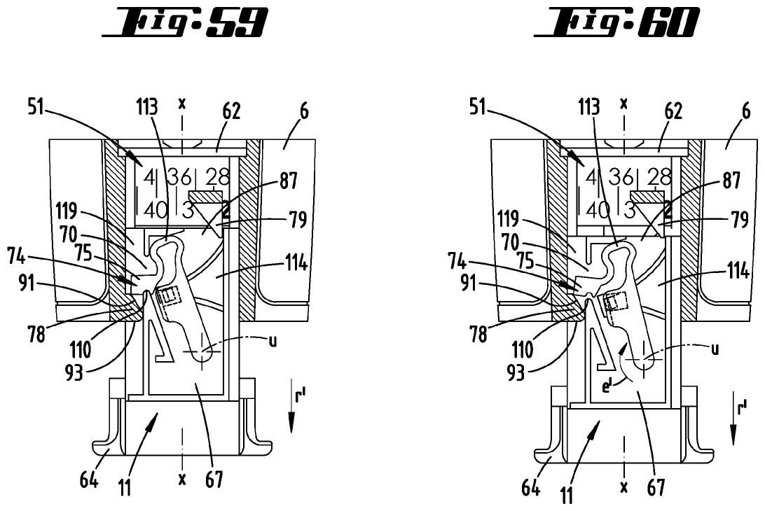

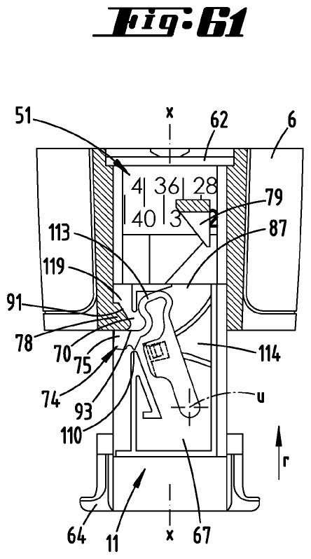

As shown in FIG. 50, during an attempt to insert an already used container 2 with the insertion locking part 74 moved into the activated position, a blocking surface 92 of the latch section 75 running essentially transversely to the insertion direction r comes into contact with a housing blocking surface 93 running essentially in this position, namely preferably parallel thereto, and aligned correspondingly transversely to the housing longitudinal axis x, which in the embodiment example shown is formed on the underside of the obstacle projection 78. In this case, the insertion locking part 74 is loaded via the obstacle projection 78 in a direction beyond the active position, which is, however, absorbed by supporting the insertion locking part 74, in particular the latch section 75, on a facing opening edge 110 of the passage opening 70. Accordingly, the insertion locking part 74 is blocked in a direction beyond the activated position. The insertion of a container 2 with an activated insertion locking part 74, correspondingly the reinsertion of an already used container 2, is thus prevented.

In the activated position, a substantially form-fitting support of the insertion locking part 74 or of the latch section 75 on the edge of the opening 110 may also result. For example, the latch section 75 facing away from the blocking surface 92 can have a recess 121 adapted to the shape (compare FIG. 44).

The user can also recognize a container 2 that has already been used by means of the insertion locking section 74 emerging through the passage openings 70 and beyond the opening plane E. Further, for this purpose, the latch section 75 can be provided with a signal color (for example, a red color) or, alternatively or in addition thereto, for example, also with a written indication (for example, “empty”).

Figures from 51 to 61 show another embodiment with respect to the activation of the insertion locking portion 74 described above.

In this embodiment, the section applying the spring force is given as a spring arm 113 formed directly by a portion of the insertion latch part 74, at the free end of which the latch section 75 described above is arranged.

The plate part-side latching projection 84 for cooperating with the latching receptacle 90 of the insertion locking part 74 is formed on a spring portion 114 of the plate part 67, which spring portion 114 does not permit resilient deflecting movement of the insertion locking part 74 in the rotational direction e or in the deflecting direction e′. Rather, the spring portion 114 is solely adapted to allow the latching projection 84 to resiliently deflect to overrun the locking shoulder 89 on the insertion locking part 74 substantially in alignment with the pivot axis u of the insertion locking part 74.

The mode of operation is similar to the previously described embodiment. When a new, correspondingly unused container 2 is inserted into the housing 4, the insertion locking part 74 is displaced about the pivot axis u in the direction of rotation e via the control projection 77 on the housing side until it reaches a detent position between the latching projection 84 and the latching receptacle 90. In the course of this controlled displacement, the control projection 77 first acts on the section of the insertion locking part 74 having the spring arm 113 and only then on a rigid section following in the pivoting direction, which has the latching receptacle 90 on the underside. In this latched activated position of the insertion locking part 74, the latch section 75 advances through the passage opening 70 and beyond the opening plane E in a latching manner into the passage gap 119. In the course of overrunning the housing-side obstacle projection 78, the latch section 75 can plunge back into the passage opening 70 in the evasion direction e′ via the spring arm 113, in order to automatically spring-assistedly advance back into the active position upon completion of the overrun. In the event of an attempt to insert an already used container 2 into the housing 4, the blocking surface 92 of the latch section 75 also steps against a housing blocking surface 93 here, whereby further the latch section 75 can support itself on the facing edge of the passage opening 70, if necessary with a recess 121, so that a displacement of the latch section 75 beyond the activated position is blocked.

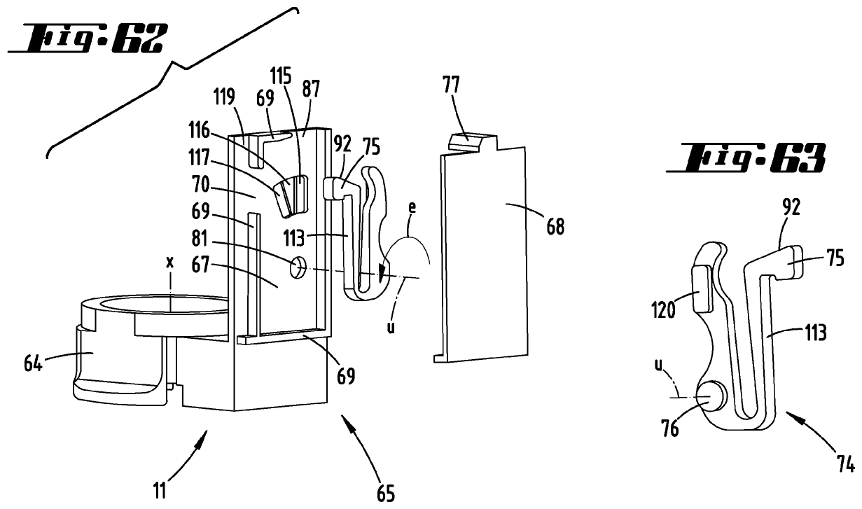

FIGS. 62 and 63 schematically show a further embodiment in which a pivotally displaceable insertion locking part 74 is also provided, with a spring arm 113 formed directly on the insertion locking part 74. In the region of the non-resiliently formed area, the insertion locking part 74 has a projection 120 on the underside which engages in a recess 115 of the plate part 67 in the non-activated position. With corresponding loading by the control projection 77 during an insertion of the container into the housing 4, a pivoting displacement of the insertion locking part 74 in the direction of rotation e also results here, with the projection 120 falling into a latching receptacles 117, which is spaced apart from the recess 115 in the direction of rotation, while overrunning a latching projection 116. In this position, the activated position is reached. The locking results substantially as illustrated with reference to the above-described embodiment.

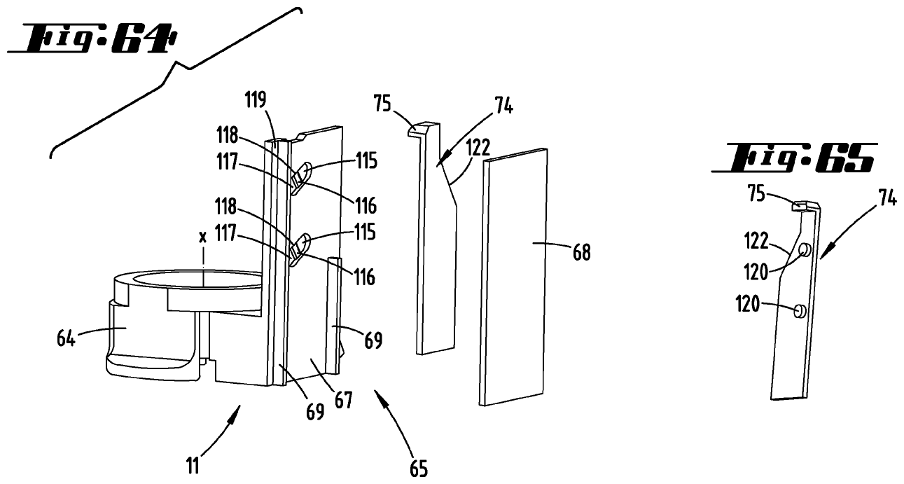

Also, according to the illustration in FIGS. 64 and 65, the housing-side control projection 77 can cooperate with a control slope 122 of the insertion locking part 74, according to which cooperation a linear displacement of the insertion locking part 74 as a whole is effected. This linear displacement can be given at an acute angle to the insertion direction r, wherein projections 120 provided on the underside of the insertion locking part 74 are also brought into latching receptacles 117 while overrunning latching projections 116 formed in inclined grooves 118, so as to achieve an activated position of the insertion locking part 74 in which it enters a blocking position with respect to a housing-side obstacle projection 78.

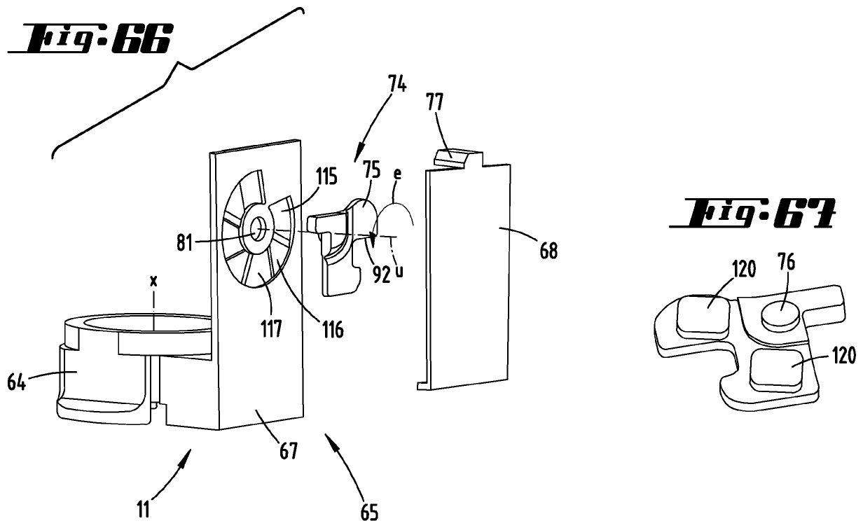

FIGS. 66 and 67 show a further embodiment in which the insertion locking part 74 is provided with projections 120 on the underside, the insertion locking part 74 further being movable about a pivot axis u in the course of the insertion movement and, in the course of such pivoting, the projections 120 entering latching receptacles 117 while overcoming latching projections 116. The overrunning of the obstacle projection 78 in the course of the removal of the container 2 from the housing 4 can lead to a further rotational displacement of the insertion locking part 74, after which finally the latch section 75 is brought into the activated, locking position.

In the embodiments of FIGS. 64 to 67, the activated position is not reached until or after the obstacle projection 78 has passed.

It is further preferred that the nebulizer 1 is provided for successive use of a maximum of three to eight, further preferred about four to six, so further if necessary five containers 2.

### Reference List

1 Nebulizer 28 Spiral inclined surface
2 Container 29 Counter inclined surface
3 Fluid 30 Piercing needle
4 Housing 31 Trigger ring
5 Upper housing part 32 Trigger button
6 Lower housing part 33 Window
7 Mouthpiece 34 Wall
8 Container wall 35 Receptacle
9 Container bottom 36 Control projection
10 Cap 37 Counter control projection
11 Container connection part 38 Recess
12 Suction tube part 39 Aerosol
13 Latching protrusion 40 Ventilation opening
14 Locking groove 41 Nozzle body
15 Cover cap 42 Upper housing inner part
16 Counter 43 Transmission part
17 Opening 44 Transmission shaft
18 Spring plate member 45 Niche
19 Holder 46 First support flange
20 Suction tube 47 Flat side
21 Passage opening 48 Passage opening
22 Non-return valve 49 First toothed formation
23 Pressure chamber 50 Second toothed formation
24 Ejection nozzle 51 Sleeve part
25 Lower housing inner part 52 Outer surface
26 Compression spring 53 Characters
27 Base part 54 Guide groove
55 Second toothing 84 Latching projection
56 Viewing window 85 Evasion space
57 First toothing 86 Groove
58 Locking projection 87 Window
59 Locking pawl 88 Slope
60 Shoulder 89 Locking shoulder
61 Spring 90 Latching receptacle
62 Second support flange 91 Control slope
63 Locking opening 92 Blocking surface
64 Base section 93 Housing blocking surface
65 Cantilever 94 Latching projection
66 Guide receptacle 95 Latching section
67 Plate part 96 Counter latching section
68 Plate part 97 Driving finger
69 Bridge 98 Counting finger
70 Passage opening 99 Wall end section
71 Return locking finger 100 Driver
72 Latching projections 101 Tooth gap
73 Control surface 102 Tooth
74 Insertion locking part 103 Locking shoulder
75 Latch section 104 Bottom section
76 Pin 105 Counter inclined surface
77 Control projection 106 Control section
78 Obstacle projection 107 Guide
79 Projection 108 Interruption area
80 Counter latching area 109 Control slope
81 Bearing opening 110 Opening edge
82 Spring arm 111 Bag
83 Attachment area 112 Receiving space
113 Spring arm
114 Spring portion
115 Recess a Direction of rotation
116 Latching projection b Direction of rotation
117 Latching receptacle b′ Direction of rotation
118 Inclined groove c Sleeve axis
119 Passage gap d Thickness
120 Projection e Direction of rotation
121 Recess e′ Deflecting direction
122 Control slope h Height
   h′ Height
   r Insertion direction
   r′ Removal direction
   u Pivot axis
   x Longitudinal axis of housing
   y Pivot axis
   z Axis of rotation
   E Opening plane
   S Locking device

1. Nebulizer (1) for a fluid (3) located in a container (2), with a housing (4), wherein the housing (4) has a mouthpiece (7) and a receiving space (112) for the container (2), which container (2) can be inserted replaceably into the housing (4), wherein furthermore for the preparation of an atomizing process a lower housing part (6) being rotatable relative to an upper housing part (5) having the mouthpiece (7), a counter (16) being furthermore provided in the lower housing part (6) for counting atomization processes carried out with an inserted container (2), and the rotation of the housing parts (5, 6) can be used to count the atomization operations, the counter (16) having a sleeve part (51) which is provided on an outer surface (52) with characters (53) representing counted atomization operations and with a toothing (57) for toothing engagement, there further being provided a transmission part (43) engaging in this toothing (57), characterized in that the toothing engagement is given in transverse direction to a sleeve axis (c) and that the transmission part (43) is arranged and formed for direct cooperation with both the upper housing part (5) and the lower housing part (6) as well as with the sleeve part (51).

2. Nebulizer according to claim 1, characterized in that the transmission part (43) is formed as a transmission shaft (44).

3. Nebulizer according to one of the preceding claims, characterized in that the transmission shaft (44) has regions with first toothed formations (49) and second toothed formations (50) spaced apart from one another in two directions of its axis of rotation (z).

4. Nebulizer according to any one of the preceding claims, characterized in that the first toothed formation (49) is designed to cooperate with the upper and lower housing parts (5, 6) and the second toothed formation (50) is designed to cooperate with the sleeve part (51).

5. Nebulizer (1) for a fluid (3) located in a container (2), with a housing (4), wherein the housing (4) has a mouthpiece (7) and a receiving space (112) for the container (2), which container (2) can be exchangeably inserted into the housing (4), wherein further for the preparation of an atomizing process a lower housing part (6) is rotatable relative to an upper housing part (5) having the mouthpiece (7), wherein further a counter (16) is provided for counting atomizing processes performed with an inserted container (2), wherein further the counter (16) comprises a sleeve part (51) provided on an outer surface (52) with characters (53) representing counted atomizing operations, or nebulizer (1) according to claims 1 to 4, characterized in that the sleeve part (51) cooperates with a locking projection (58) which is guided in the upper housing part (5) in a manner fixed to the direction of rotation and which, in a selected position of the sleeve part (51), moves into a locking opening (63) of the sleeve part (51).

6. Nebulizer of claim 5, characterized in that the lower housing member (6) includes a circumferential first support flange (46) for the locking projection (58), the support flange (46) leaving a passage opening (48) for the locking projection (58) in a selected circumferential region.

7. Nebulizer of any one of claims 5 or 6, characterized in that the sleeve part (51) includes a second support flange (62) having the locking opening (63) for the locking projection (58) in a selected peripheral region thereof.

8. Nebulizer (1) for a fluid (3) located in a container (2), having a housing (4), the housing (4) having a mouthpiece (7) and a receiving space (112) for the container (2), which container (2) can be inserted replaceably into the housing (4), a container (2) removed from the housing (4) no longer being able to be moved into a position of use, or nebulizer (1) according to one of the claims 1 to 7, characterized in that the container (2) is provided with a fixedly connected cantilever (65) and in that the cantilever (65), in the course of insertion of the container (2) into the housing (4), cooperates with a projection (79) fixed to the housing to displace an insertion locking part (74) into an activated position.

9. Nebulizer according to claim 8, characterized in that the insertion locking part (74) moves in a transverse direction to a longitudinal axis (x) of the nebulizer (1) in the course of inserting the container (2) into the housing (4).

10. Nebulizer according to any one of claims 8 or 9, characterized in that the insertion locking part (74) is rotatably mounted on the cantilever (65).

11. Nebulizer according to any one of claims 8 to 10, characterized in that at least a portion of the insertion locking part (74) is displaceable from the activated position toward a non-activated position against spring force.

12. Nebulizer according to any one of claims 8 to 11, characterized in that the insertion locking part (74) is locked beyond the activated position.

13. Nebulizer according to one of the claims 11 or 12, characterized in that the spring force is achieved by a design of the insertion locking part (74) as a spring arm (113).

14. Nebulizer according to one of the claims 11 or 12, characterized in that the spring force is achieved by a spring arm (82) formed separately from the insertion locking part (74) and lockable with the insertion locking part (74).

15. Nebulizer according to any one of claim 13, characterized in that the spring arm (82) provided separately from the insertion locking member (74) is formed on the cantilever (65).

16. Container (2) for a nebulizer (1), characterized in that the container (2) is provided with a fixedly connected cantilever (65) and that the cantilever (65) comprises an insertion locking part (74).

17. Container according to claim 16, characterized in that the insertion locking part (74) is displaceable to a locking position.

18. Container of claim 17, further comprising any of the features of the insertion latch member (74) of any of claims 9 to 15.
